SEQUENCE LISTING (1 sequence)

SEQ ID NO: 1 - DNA, 14 bp, Artificial
  human matrix metalloproteinase-1 targeted artificial sequence
  misc_feature (1)..(1): Carbamated N-terminal
  misc_feature (1)..(14): PNA modified olegonucleotide
  modified_base (2)..(2): n is a, g, c, or t
  modified_base (6)..(6): n is a, g, c, or t
  modified_base (8)..(8): n is a, g, c, or t
  modified_base (9)..(9): n is a, g, c, or t
  modified_base (11)..(11): n is a, g, c, or t
  modified_base (13)..(13): n is a, g, c, or t
tnctcncnntntnt

(19) United States
(12) Patent Application Publication
(10) Pub. No.: US 2021/0292369 A1
(43) Pub. Date: Sep. 23, 2021

(54) MATRIX METALLOPROTEINASE-1 ANTISENSE OLIGONUCLEOTIDES

(71) Applicant: OLIPASS CORPORATION, Gyeonggi-do (KR)

(72) Inventors: Seon-Young HAN, Gangwon-do (KR); Kiho SUNG, Gyeonggi-do (KR); Myunghyo HONG, Gyeonggi-do (KR); Youree OH, Gyeonggi-do (KR); Jeong-Seok HEO, Gyeonggi-do (KR); Kang Won JANG, Gyeonggi-do (KR)

(21) Appl. No.: 17/055,809

(22) PCT Filed: May 3, 2019
(86) PCT No.: PCT/KR2019/005994

§ 371 Date: Nov. 16, 2020

(30) Foreign Application Priority Data
May 18, 2018 (KR) 10-2018-0057352

(51) Int. Cl. C07K 14/00; C12N 15/113; A61K 8/64; A61K 8/60; A61Q 19/08

(52) U.S. Cl. CPC C07K 14/003; C12N 15/1137; A61K 8/64; C12N 2320/30; A61Q 19/08; C12N 2310/11; C12N 2310/3181; A61K 8/606

(57) ABSTRACT

A method to treat diseases or conditions associated with the human MMP-1 gene transcription involving administration of the peptide nucleic acid derivative according to claim 1 to a subject. The present invention provides the peptide nucleic acid derivative according to claim 1 which targets 5′ splice site of the human MMP-1 pre-mRNA “exon 5”. The peptide nucleic acid derivatives in the present invention strongly induce splice variants of the human MMP-1 mRNA in cell and are very useful to treat conditions or diseases of skin aging associated with the human MMP-1 protein.

This application is a national stage application under 35 U.S.C. § 371 of International Application No. PCT/KR2019/005994, filed on May 3, 2019, which claims priority of Korean Application No. 10-2018-0057352, filed on May 18, 2018, which are hereby incorporated by reference in their entirety.

### TECHNICAL FIELD

This invention relates to peptide nucleic acid derivatives complementarily targeting the human matrix metalloproteinase-1 pre-mRNA for improvement of skin aging mediated by matrix metalloproteinase-1.

### BACKGROUND ART

Skin aging has received considerable attention since the signs of aging are most visible in the skin. As the prevention and treatment for skin aging is very important in quality of life, the already aged as well as the youth are interested in related health food, cosmetics, medicine, medical supplies, and so on. There are two primary skin aging processes. One is intrinsic or natural aging that accompany aging and the other is extrinsic aging, which is caused by exogenous origin such as solar exposure, smoking, and malnutrition.

The ultraviolet irradiation exposure on the skin accelerates expression of matrix metalloproteinase-1 (MMP-1) to give rise to promote the degradation of collagen, the primary structural component of the dermis. In addition, smoking induces matrix metalloproteinase-1 mRNA in the skin, which causes same results as the ultraviolet irradiation exposure on the skin [J. Cosmetic Dermatology vol 6, 40-50 (2007)].

Collagen is the main structural protein in the extracellular space in the various connective tissues such as skin, blood vessel, bone, tooth, and muscle. Since collagen is responsible for supporting most tissues and cells structure, its degradation and deformation may strongly affect skin aging [J. Pathol. vol 211, 241-251 (2007)].

Matrix metalloproteinases are enzymes that are secreted from fibroblast, keratinocyte, and so on, which are capable of degrading all kinds of extracellular matrix (ECM) and basement membrane (BM). More than 26 kinds of matrix metalloproteinases were identified such as MMP-1 (interstitial collagenase), MMP-2 (gelatinase), MMP-3 (stromelysin), MMP-7 (Matrilysin), MMP-8 (neutrophil collagenase), and MMP-12 (metalloelastase) [J. Biol. Chem. vol 277, 451-454 (2002); J. Matrix. Biol. vol 15, 519-526 (1997)].

Reactive oxygen species caused by ultraviolet irradiation exposure and smoking have been known for the reason of overexpression of matrix metalloproteinase-1. In that sense antioxidants and functional food for antioxidizing effect, such as vitamin A (retinol), vitamin C (ascorbic acid), vitamin E (tocopherol), carotenoid, flavonoid, green tea, and selenium, have been developed for the treatment of skin aging and the study on the mechanism of action is currently underway [Int. J. Food Sci. Technol. vol 73, 989-996 (2005); Kor. J. Aesthet. Cosmetol. vol 11, 649-654 (2013); Int. J. Mol. Med. vol 38, 357-363 (2016)].

Considering the significance of metalloproteinase-1 in skin aging, it is very interesting and necessary to develop the pharmaceuticals or cosmetics based on the mechanism of metalloproteinase-1 expression, which may improve and prevent skin aging condition.

Pre-mRNA:

Genetic information is carried on DNA (2-deoxyribose nucleic acid). DNA is transcribed to produce pre-mRNA (pre-messenger ribonucleic acid) in the nucleus. Mammalian pre-mRNA usually consists of exons and introns, and exon and intron are interconnected to each other as schematically provided. Exons and introns are numbered as exemplified in FIG. 14.

Splicing of Pre-mRNA:

Pre-mRNA is processed into mRNA following deletion of introns by a series of complex reactions collectively called “splicing” which is schematically summarized in FIG. 15 [Ann. Rev. Biochem. 72(1), 291-336 (2003); Nature Rev. Mol. Cell Biol. 6(5), 386-398 (2005); Nature Rev. Mol. Cell Biol. 15(2), 108-121 (2014)].

Splicing is initiated by forming “spliceosome E complex” (i.e. early spliceosome complex) between pre-mRNA and splicing adapter factors. In “spliceosome E complex”, U1 binds to the junction of exon N and intron N, and U2AF³⁵ binds to the junction of intron N and exon (N+1). Thus the junctions of exon/intron or intron/exon are critical to the formation of the early spliceosome complex. “Spliceosome E complex” evolves into “spliceosome A complex” upon additional complexation with U2. The “spliceosome A complex” undergoes a series of complex reactions to delete or splice out the intron to adjoin the neighboring exons.

Ribosomal Protein Synthesis:

Proteins are encoded by DNA (2-deoxyribose nucleic acid). In response to cellular stimulation or spontaneously, DNA is transcribed to produce pre-mRNA (pre-messenger ribonucleic acid) in the nucleus. The introns of pre-mRNA are enzymatically spliced out to yield mRNA (messenger ribonucleic acid), which is then translocated into the cytoplasm. In the cytoplasm, a complex of translational machinery called ribosome binds to mRNA and carries out the protein synthesis as it scans the genetic information encoded along the mRNA [Biochemistry vol 41, 4503-4510 (2002); Cancer Res. vol 48, 2659-2668 (1988)].

Antisense Oligonucleotide (ASO):

An oligonucleotide binding to nucleic acid including DNA, mRNA and pre-mRNA in a sequence specific manner (i.e. complementarily) is called antisense oligonucleotide (ASO).

If an ASO tightly binds to an mRNA in the cytoplasm, for example, the ASO may be able to inhibit the ribosomal protein synthesis along the mRNA. ASO needs to be present within the cytoplasm in order to inhibit the ribosomal protein synthesis of its target protein.

Antisense Inhibition of Splicing:

If an ASO tightly binds to a pre-mRNA in the nucleus, the ASO may be able to inhibit or modulate the splicing of pre-mRNA into mRNA. ASO needs to be present within the nucleus in order to inhibit or modulate the splicing of pre-mRNA into mRNA. Such antisense inhibition of splicing produces an mRNA or mRNAs lacking the exon targeted by the ASO. Such mRNA(s) is called “splice variant(s)”, and encodes protein(s) smaller than the protein encoded by the full-length mRNA.

In principle, splicing can be interrupted by inhibiting the formation of “spliceosome E complex”. If an ASO tightly binds to a junction of (5′→3′) exon-intron, i.e. “5′ splice site”, the ASO blocks the complex formation between pre-mRNA and factor U1, and therefore the formation of “spliceosome E complex”. Likewise, “spliceosome E complex” cannot be formed if an ASO tightly binds to a junction of (5′→3′) intron-exon, i.e. “3′ splice site”.

3′ splice site and 5′ splice site are schematically illustrated in FIG. 16.

Unnatural Oligonucleotides:

DNA or RNA oligonucleotides are susceptible to degradation by endogenous nucleases, limiting their therapeutic utility. To date, many types of unnatural (naturally non-occurring) oligonucleotides have been developed and studied intensively [Clin. Exp. Pharmacol. Physiol. vol 33, 533-540 (2006)]. Some of them show extended metabolic stability compared to DNA and RNA. Provided below are the chemical structures for a few of representative unnatural oligonucleotides. Such oligonucleotides predictably bind to a complementary nucleic acid as DNA or RNA does.

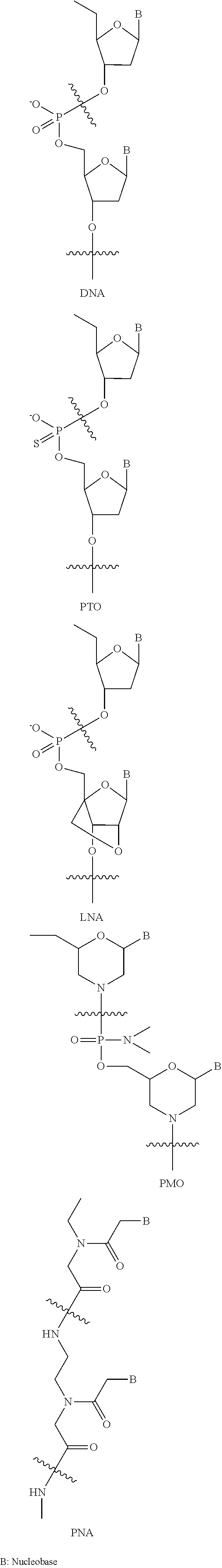

Phosphorothioate Oligonucleotide:

Phosphorothioate oligonucleotide (PTO) is a DNA analog with one of the backbone phosphate oxygen atoms replaced with a sulfur atom per monomer. Such a small structural change made PTO comparatively resistant to degradation by nucleases [Ann. Rev. Biochem. vol 54, 367-402 (1985)].

Reflecting the structural similarity in the backbone of PTO and DNA, they both poorly penetrate the cell membrane in most mammalian cell types. For some types of cells abundantly expressing transporter(s) of DNA, however, DNA and PTO show good cell penetration. Systemically administered PTOs are known to readily distribute to the liver and kidney [Nucleic Acids Res. vol 25, 3290-3296 (1997)].

In order to facilitate PTO's cell penetration in vitro, lipofection has been popularly practiced. However, lipofection physically alters the cell membrane, causes cytotoxicity, and therefore would not be ideal for long term in vivo therapeutic use.

Over the past 30 years, antisense PTOs and variants of PTOs have been clinically evaluated to treat cancers, immunological disorders, metabolic diseases, and so on [Biochemistry vol 41, 4503-4510 (2002); Clin. Exp. Pharmacol. Physiol. vol 33, 533-540 (2006)]. Many of such antisense drug candidates have not been successfully developed partly due to PTO's poor cell penetration. In order to overcome the poor cell penetration, PTO needs to be administered at high dose for therapeutic activity. However, PTOs are known to be associated with dose-limiting toxicity including increased coagulation time, complement activation, tubular nephropathy, Kupffer cell activation, and immune stimulation including splenomegaly, lymphoid hyperplasia, mononuclear cell infiltration [Clin. Exp. Pharmacol. Physiol. vol 33, 533-540 (2006)].

Many antisense PTOs have been found to show due clinical activity for diseases with a significant contribution from the liver or kidney. Mipomersen is a PTO analog which inhibits the synthesis of apoB-100, a protein involved in LDL cholesterol transport. Mipomersen manifested due clinical activity in atherosclerosis patients most likely due to its preferential distribution to the liver [Circulation vol 118(7), 743-753 (2008)]. ISIS-113715 is a PTO antisense analog inhibiting the synthesis of protein tyrosine phosphatase 1B (PTP1B), and was found to show therapeutic activity in type II diabetes patients. [Curr. Opin. Mol. Ther. vol 6, 331-336 (2004)].

Locked Nucleic Acid:

In locked nucleic acid (LNA), the backbone ribose ring of RNA is structurally constrained to increase the binding affinity for RNA or DNA. Thus, LNA may be regarded as a high affinity DNA or RNA analog [Biochemistry vol 45, 7347-7355 (2006)].

Phosphorodiamidate Morpholino Oligonucleotide:

In phosphorodiamidate morpholino oligonucleotide (PMO), the backbone phosphate and 2-deoxyribose of DNA are replaced with phosphoramidate and morpholine, respectively [Appl. Microbiol. Biotechnol. vol 71, 575-586 (2006)]. Whilst the DNA backbone is negatively charged, the PMO backbone is not charged. Thus the binding between PMO and mRNA is free of electrostatic repulsion between the backbones, and tends to be stronger than that between DNA and mRNA. Since PMO is structurally very different from DNA, PMO wouldn't be recognized by the hepatic transporter(s) recognizing DNA or RNA. Nevertheless, PMO doesn't readily penetrate the cell membrane.

Peptide Nucleic Acid:

Peptide nucleic acid (PNA) is a polypeptide with N-(2-aminoethyl)glycine as the unit backbone, and was discovered by Dr. Nielsen and colleagues [Science vol 254, 1497-1500 (1991)]. The chemical structure and abbreviated nomenclature of PNA are illustrated below. Like DNA and RNA, PNA also selectively binds to complementary nucleic acid. [Nature (London) vol 365, 566-568 (1992)]. In binding to complementary nucleic acid, the N-terminus of PNA is regarded as equivalent to the “5′-end” of DNA or RNA, and the C-terminus of PNA as equivalent to the “3′-end” of DNA or RNA.

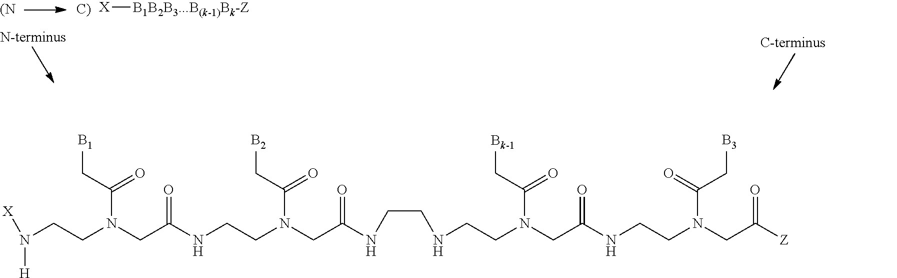

Like PMO, the PNA backbone is not charged. Thus the binding between PNA and RNA tends to be stronger than the binding between DNA and RNA. Since PNA is markedly different from DNA in the chemical structure, PNA wouldn't be recognized by the hepatic transporter(s) recognizing DNA, and would show a tissue distribution profile different from that of DNA or PTO. However, PNA also poorly penetrates the mammalian cell membrane [Adv. Drug Delivery Rev. vol 55, 267-280 (2003)].

Modified Nucleobases to Improve Membrane Permeability of PNA:

PNA was made highly permeable to mammalian cell membrane by introducing modified nucleobases with a cationic lipid or its equivalent covalently attached thereto. The chemical structures of such modified nucleobases are provided below. Such modified nucleobases of cytosine, adenine, and guanine were found to predictably and complementarily hybridize with guanine, thymine, and cytosine, respectively [PCT Appl. No. PCT/KR2009/001256; EP2268607; U.S. Pat. No. 8,680,253].

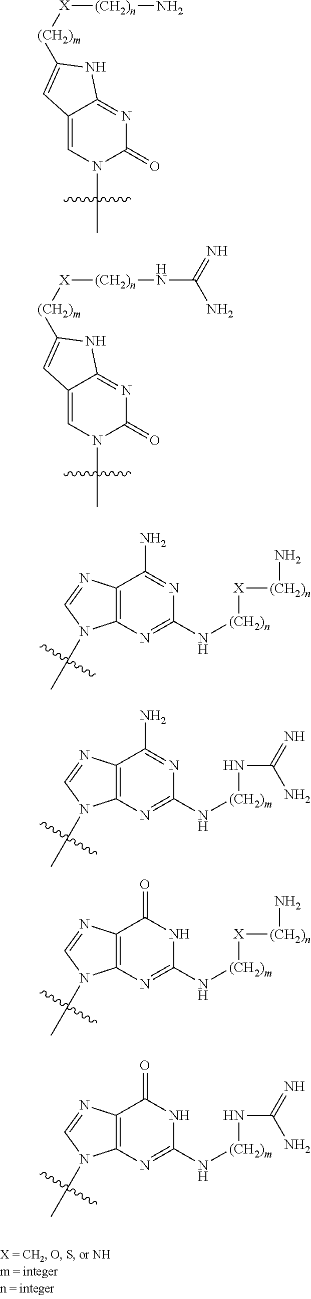

Incorporation of such modified nucleobases onto PNA resembles situations of lipofection. By lipofection, oligonucleotide molecules with phosphate backbone are wrapped with cationic lipid molecules such as lipofectamine, and such lipofectamine/oligonucleotide complexes tend to penetrate membrane rather easily as compared to naked oligonucleotide molecules.

In addition to good membrane permeability, those PNA derivatives were found to possess ultra-strong affinity for complementary nucleic acid. For example, introduction of 4 to 5 modified nucleobases onto 11- to 13-mer PNA derivatives easily yielded a Tₘ gain of 20° C. or higher in duplex formation with complementary DNA. Such PNA derivatives are highly sensitive to a single base mismatch. A single base mismatch resulted in a Tₘ loss of 11 to 22° C. depending on the type of modified base as well as PNA sequence.

Small Interfering RNA (siRNA):

Small interfering RNA (siRNA) refers to a double stranded RNA of 20-25 base pairs [Microbiol. Mol. Biol. Rev. vol 67(4), 657-685 (2003)]. The anti sense strand of siRNA somehow interacts with proteins to form an “RNA-induced Silencing Complex” (RISC). Then the RISC binds to a certain portion of mRNA complementary to the antisense strand of siRNA. The mRNA complexed with the RISC undergoes cleavage. Thus siRNA catalytically induces the cleavage of its target mRNA, and consequently inhibits the protein expression by the mRNA. The RISC does not always bind to the full complementary sequence within its target mRNA, which raises concerns relating to off-target effects of an siRNA therapy. Like other classes of oligonucleotide with DNA or RNA backbone, siRNA possesses poor cell permeability and therefore tends to show poor in vitro or in vivo therapeutic activity unless properly formulated or chemically modified to have good membrane permeability.

Matrix Metalloproteinase-1 siRNA:

A MMP-1 siRNA targeting a 19-mer RNA sequence within the human MMP-1 mRNA was reported to inhibit the expression of the MMP-1 mRNA and protein in human chondrosarcoma following a lipofection at 100 nM [J. Orthop. Res. vol 23, 1467-1474 (2005)]. This result may be useful to the study of MMP-1 related cancer metastasis and the development of cancer therapeutics.

### DISCLOSURE OF THE INVENTION

### Problem to be Solved

Antioxidants and functional food for antioxidizing effect have been developed for the treatment of skin aging with limited efficacy and the study on the mechanism of action is currently underway. Although MMP-1 related siRNA was reported to inhibit the expression of the MMP-1 mRNA and protein in vitro, siRNAs are too expensive to manufacture and they need to be delivered into the skin by trans-dermal administration for good user compliance. Therefore, considering the significance of metalloproteinase-1 in skin aging, it is very interesting and necessary to develop the pharmaceuticals and cosmetics based on the mechanism of metalloproteinase-1 expression, which may improve and prevent skin aging condition.

### Solution to the Problem

The present invention provides a peptide nucleic acid derivative represented by Formula I, or a pharmaceutically acceptable salt thereof:

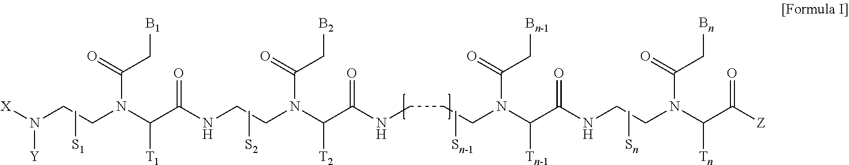

wherein,

n is an integer between 10 and 21;

the compound of Formula I possesses at least a 10-mer complementary overlap with the 16-mer pre-mRNA sequence of [(5′→3′) CAUAUAUGGUGAGUAU] in the human MMP-1 pre-mRNA;

the compound of Formula I is fully complementary to the human MMP-1 pre-mRNA, or partially complementary to the human MMP-1 pre-mRNA with one or two mismatches;

S₁, S₂, . . . , Sₙ₋₁, Sₙ, T₁, T₂, . . . , Tₙ₋₁, and Tₙ independently represent hydrido, deuterido, substituted or non-substituted alkyl, or substituted or non-substituted aryl radical;

X and Y independently represent hydrido, deuterido, formyl [H—C(═O)—], aminocarbonyl [NH₂—C(═O)—], aminothiocarbonyl [NH₂—C(═S)—], substituted or non-substituted alkyl, substituted or non-substituted aryl, substituted or non-substituted alkyloxy, substituted or non-substituted aryloxy, substituted or non-substituted alkylacyl, substituted or non-substituted arylacyl, substituted or non-substituted alkyloxycarbonyl, substituted or non-substituted aryloxycarbonyl, substituted or non-substituted alkylaminocarbonyl, substituted or non-substituted arylaminocarbonyl, substituted or non-substituted alkylaminothiocarbonyl, substituted or non-substituted arylaminothiocarbonyl, substituted or non-substituted alkyloxythiocarbonyl, substituted or non-substituted aryloxythiocarbonyl, substituted or non-substituted alkylsulfonyl, substituted or non-substituted arylsulfonyl, substituted or non-substituted alkylphosphonyl, or substituted or non-substituted arylphosphonyl radical;

Z represents hydrido, deuterido, hydroxy, substituted or non-substituted alkyloxy, substituted or non-substituted aryloxy, substituted or non-substituted amino, substituted or non-substituted alkyl, or substituted or non-substituted aryl radical;

B₁, B₂, . . . , Bₙ₋₁, and Bₙ are independently selected from natural nucleobases including adenine, thymine, guanine, cytosine and uracil, and unnatural nucleobases; and, at least four of B₁, B₂, . . . , Bₙ₋₁, and Bₙ are independently selected from unnatural nucleobases with a substituted or non-substituted amino radical covalently linked to the nucleobase moiety.

The compound of Formula I induces the skipping of “exon 5” in the human MMP-1 pre-mRNA, yields the human MMP-1 mRNA splice variant(s) lacking “exon 5”, and therefore is useful to inhibit the functional activity of the gene transcribing the human MMP-1 pre-mRNA.

The condition that “n is an integer between 10 and 21” literally means that n is an integer selectable from a group of integers of 11, 12, 13, 14, 15, 16, 17, 18, 19, and 20.

The chemical structures of natural or unnatural nucleobases in the PNA derivative of Formula I are exemplified in FIGS. 1a-1c. Natural (i.e. naturally occurring) or unnatural (naturally non-occurring) nucleobases of this invention comprise but are not limited to the nucleobases provided in FIGS. 1a-1c. Provision of such unnatural nucleobases is to illustrate the diversity of allowable nucleobases, and therefore should not be interpreted to limit the scope of the present invention.

The substituents adopted to describe the PNA derivative of Formula I are exemplified in FIGS. 2a-2e. FIG. 2a provides examples for substituted or non-substituted alkyl radicals. Substituted or non-substituted alkylacyl and substituted or non-substituted arylacyl radicals are exemplified in FIG. 2b. FIG. 2c illustrates examples for substituted or non-substituted alkylamino, substituted or non-substituted arylamino, substituted or non-substituted aryl, substituted or non-substituted alkylsulfonyl or arylsulfonyl, and substituted or non-substituted alkylphosphonyl or arylphosphonyl radicals. FIG. 2d provides examples for substituted or non-substituted alkyloxycarbonyl or aryloxycarbonyl, substituted or non-substituted alkyl aminocarbonyl or arylaminocarbonyl radicals. In FIG. 2e are provided examples for substituted or non-substituted alkylaminothiocarbonyl, substituted or non-substituted arylaminothiocarbonyl, substituted or non-substituted alkyloxythiocarbonyl, and substituted or non-substituted aryloxythiocarbonyl radicals. Provision of such exemplary substituents is to illustrate the diversity of allowable substituents, and therefore should not be interpreted to limit the scope of the present invention. A skilled person in the field may easily figure out that oligonucleotide sequence is the overriding factor for sequence specific binding of oligonucleotide to the target pre-mRNA sequence over substituents in the N-terminus or C-terminus.

The compound of Formula I tightly binds to the complementary DNA as exemplified in the prior art [PCT/KR2009/001256]. The duplex between the PNA derivative of Formula I and its full-length complementary DNA or RNA possesses a Tₘ value too high to be reliably determined in aqueous buffer. The PNA compound of Formula I yields high Tₘ values with complementary DNAs of shorter length.

The compound of Formula I complementarily binds to the 5′ splice site of “exon 5” of the human MMP-1 pre-mRNA. [NCBI Reference Sequence: NG_011740]. The 16-mer sequence of [(5′→3′) CAUAUAUGGUGAGUAU] spans the junction of “exon 5” and “intron 5” in the human MMP-1 pre-mRNA, and consists of 8-mer from “exon 5” and 8-mer from “intron 5”. Thus the 16-mer pre-mRNA sequence may be conventionally denoted as [(5′→3′) CAUAUAUG|gugaguau], wherein the exon and intron sequence are provided as “capital” and “small” letters, respectively, and the exon-intron junction is expressed with “|”. The conventional denotation for pre-mRNA is further illustrated by a 30-mer sequence of [(5′→3′) UCCAAGCCAUAUAUG|gugaguauggggaaa] spanning the junction of “exon 5” and “intron 5” in the human MMP-1 pre-mRNA.

The compound of Formula I tightly binds to the target 5′ splice site of the human MMP-1 pre-mRNA transcribed from the human MMP-1 gene, and interferes with the formation of “spliceosome early complex” to yield MMP-1 mRNA splice variant(s) lacking “exon 5” (exon 5 skipping).

The strong RNA affinity allows the compound of Formula I to induce the skipping of MMP-1 “exon 5”, even when the PNA derivative possesses one or two mismatches with the target 5′ splice site in the MMP-1 pre-mRNA. Similarly the PNA derivative of Formula I may still induce the skipping of MMP-1 “exon 5” in a MMP-1 mutant pre-mRNA possessing one or two SNPs (single nucleotide polymorphism) in the target splice site.

The compound of Formula I possesses good cell permeability and can be readily delivered into cell as “naked” oligonucleotide as exemplified in the prior art [PCT/KR2009/001256]. Thus the compound of this invention induces the skipping of “exon 5” in the MMP-1 pre-mRNA, and yields MMP-1 mRNA splice variant(s) lacking MMP-1 “exon 5” in cells treated with the compound of Formula I as “naked” oligonucleotide. The compound of Formula I does not require any means or formulations for delivery into cell to potently induce the skipping of the target exon in cells. The compound of Formula I readily induces the skipping of MMP-1 “exon 5” in cells treated with the compound of this invention as “naked” oligonucleotide at sub-femtomolar concentration.

Owing to the good cell or membrane permeability, the PNA derivative of Formula I can be topically administered as “naked” oligonucleotide to induce the skipping of MMP-1 “exon 5” in the skin. The compound of Formula I does not require a formulation to increase trans-dermal delivery into target tissue for the intended therapeutic or biological activity. Usually the compound of Formula I is dissolved in water and co-solvent, and topically or trans-dermally administered at subpicomolar concentration to elicit the desired therapeutic or biological activity in target skin. The compound of this invention does not need to be heavily or invasively formulated to elicit the topical therapeutic activity. Nevertheless, the PNA derivative of Formula I can be formulated with cosmetic ingredients or adjuvants as topical cream or lotion. Such topical cosmetic cream or lotion may be useful to treat skin aging.

The compound of the present invention can be topically administered to a subject at a therapeutically or biologically effective concentration ranging from 1 aM to higher than 1 nM, which would vary depending on the dosing schedule, conditions or situations of the subject, and so on.

The PNA derivative of Formula I can be variously formulated including but not limited to injections, nasal spray, transdermal patch, and so on. In addition, the PNA derivative of Formula I can be administered to the subject at therapeutically effective dose and the dose of administration can be diversified depending on indication, administration route, dosing schedule, conditions or situations of the subject, and so on.

The compound of Formula I may be used as combined with a pharmaceutically acceptable acid or base including but not limited to sodium hydroxide, potassium hydroxide, hydrochloric acid, methanesulfonic acid, citric acid, trifluoroacetic acid, and so on.

The PNA derivative of Formula I or a pharmaceutically acceptable salt thereof can be administered to a subject in combination with a pharmaceutically acceptable adjuvant including but not limited to citric acid, hydrochloric acid, tartaric acid, stearic acid, polyethyleneglycol, polypropyleneglycol, ethanol, isopropanol, sodium bicarbonate, distilled water, preservative(s), and so on.

Of interest is a PNA derivative of Formula I, or a pharmaceutically acceptable salt thereof:

wherein,

n is an integer between 10 and 21;

the compound of Formula I possesses at least a 10-mer complementary overlap with

the 16-mer pre-mRNA sequence of [(5′→3′) CAUAUAUGGUGAGUAU] in the human MMP-1 pre-mRNA;

the compound of Formula I is fully complementary to the human MMP-1 pre-mRNA, or partially complementary to the human MMP-1 pre-mRNA with one or two mismatches;

S₁, S₂, . . . , Sₙ₋₁, Sₙ, T₁, T₂, . . . , Tₙ₋₁, and Tₙ independently represent hydrido, deuterido radical;

X and Y independently represent hydrido, deuterido, formyl [H—C(═O)—], aminocarbonyl [NH₂—C(═O)—], aminothiocarbonyl [NH₂—C(═S)—], substituted or non-substituted alkyl, substituted or non-substituted aryl, substituted or non-substituted alkyloxy, substituted or non-substituted aryloxy, substituted or non-substituted alkylacyl, substituted or non-substituted arylacyl, substituted or non-substituted alkyloxycarbonyl, substituted or non-substituted aryloxycarbonyl, substituted or non-substituted alkylaminocarbonyl, substituted or non-substituted arylaminocarbonyl, substituted or non-substituted alkylaminothiocarbonyl, substituted or non-substituted arylaminothiocarbonyl, substituted or non-substituted alkyloxythiocarbonyl, substituted or non-substituted aryloxythiocarbonyl, substituted or non-substituted alkylsulfonyl, substituted or non-substituted arylsulfonyl, substituted or non-substituted alkylphosphonyl, or substituted or non-substituted arylphosphonyl radical;

Z represents hydrido, hydroxy, substituted or non-substituted alkyloxy, substituted or non-substituted aryloxy, or substituted or non-substituted amino radical;

B₁, B₂, . . . , Bₙ₋₁, and Bₙ are independently selected from natural nucleobases including adenine, thymine, guanine, cytosine and uracil, and unnatural nucleobases;

at least four of B₁, B₂, . . . , Bₙ₋₁, and Bₙ are independently selected from unnatural nucleobases represented by Formula II, Formula III, or Formula IV:

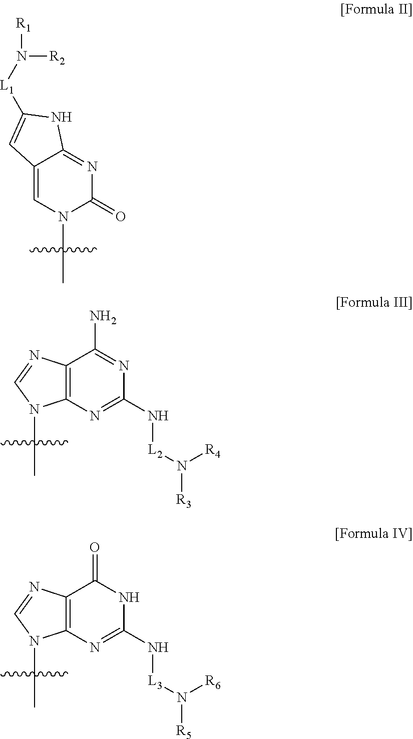

wherein,

R₁, R₂, R₃, R₄, R₅ and R₆ are independently selected from hydrido and substituted or non-substituted alkyl radical;

L₁, L₂ and L₃ are a covalent linker represented by Formula V covalently linking the basic amino group to the nucleobase moiety:

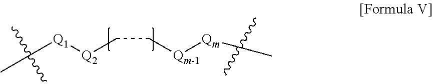

wherein,

Q₁ and Qₘ are substituted or non-substituted methylene (—CH₂—) radical, and Qₘ is directly linked to the basic amino group;

Q₂, Q₃, . . . , and Qₘ₋₁ are independently selected from substituted or non-substituted methylene, oxygen (—O—), sulfur (—S—), and substituted or non-substituted amino radical [—N(H)—, or —N(substituent)-]; and,

m is an integer between 1 and 15.

Of high interest is a PNA oligomer of Formula I, or a pharmaceutically acceptable salt thereof:

wherein,

n is an integer between 11 and 16;

the compound of Formula I possesses at least a 10-mer complementary overlap with the 16-mer pre-mRNA sequence of [(5′→3′) CAUAUAUGGUGAGUAU] in the human MMP-1 pre-mRNA;

the compound of Formula I is fully complementary to the human MMP-1 pre-mRNA;

S₁, S₂, . . . , Sₙ₋₁, Sₙ, T₁, T₂, . . . , Tₙ₋₁, and Tₙ are hydrido radical;

X and Y independently represent hydrido, substituted or non-substituted alkylacyl, or substituted or non-substituted alkyloxycarbonyl radical;

Z represents substituted or non-substituted amino radical;

B₁, B₂, . . . , Bₙ₋₁, and Bₙ are independently selected from natural nucleobases including adenine, thymine, guanine, cytosine and uracil, and unnatural nucleobases;

at least five of B₁, B₂, . . . , Bₙ₋₁, and Bₙ are independently selected from unnatural nucleobases represented by Formula II, Formula III, or Formula IV;

R₁, R₂, R₃, R₄, R₅ and R₆ are hydrido radical;

Q₁ and Qₘ are methylene radical, and Qₘ is directly linked to the basic amino group; Q₂, Q₃, . . . , and Qₘ₋₁ are independently selected from methylene and oxygen radical; and,

m is an integer between 1 and 9.

Of higher interest is a PNA derivative of Formula I, or a pharmaceutically acceptable salt thereof:

wherein,

n is an integer between 11 and 16;

the compound of Formula I possesses at least a 10-mer complementary overlap with the 16-mer pre-mRNA sequence of [(5′→3′) CAUAUAUGGUGAGUAU] in the human MMP-1 pre-mRNA;

the compound of Formula I is fully complementary to the human MMP-1 pre-mRNA;

S₁, S₂, . . . , Sₙ₋₁, Sₙ, T₁, T₂, . . . , Tₙ₋₁, and Tₙ are hydrido radical;

X is hydrido radical;

Y represents substituted or non-substituted alkyloxycarbonyl radical;

Z represents substituted or non-substituted amino radical;

B₁, B₂, . . . , Bₙ₋₁, and Bₙ are independently selected from natural nucleobases including adenine, thymine, guanine, cytosine and uracil, and unnatural nucleobases;

at least five of B₁, B₂, . . . , Bₙ₋₁, and Bₙ are independently selected from unnatural nucleobases represented by Formula II, Formula III, or Formula IV;

R₁, R₂, R₃, R₄, R₅ and R₆ are hydrido radical;

L₁ represents —(CH₂)₂—O—(CH₂)₂—, —CH₂—O—(CH₂)₂—, —CH₂—O—(CH₂)₃—, —CH₂—O—(CH₂)₄—, or —CH₂—O—(CH₂)₅— with the right end is directly linked to the basic amino group; and,

L₂ and L₃ are independently selected from —(CH₂)₂—O—(CH₂)₂—, —(CH₂)₃—O—(CH₂)₂—, —(CH₂)₂—O—(CH₂)₃—, —(CH₂)₂—, —(CH₂)₃—, —(CH₂)₄—, —(CH₂)₅—, —(CH₂)₆—, —(CH₂)₇—, and —(CH₂)₈— with the right end is directly linked to the basic amino group.

Of specific interest is a PNA derivative of Formula I which is the compound provided below (ASO 1, Antisense Oligonucleotide 1) or a pharmaceutically acceptable salt thereof:

(N→C) Fethoc-TA(6)C-TCA(6)-CC(102)A(6)-TA(6)T-A(6)T-NH₂

wherein,

A, T, and C are PNA monomers with a natural nucleobase of adenine, thymine, and cytosine, respectively;

C(pOq) and A(p) are PNA monomers with an unnatural nucleobase represented by Formula VI and Formula VII, respectively;

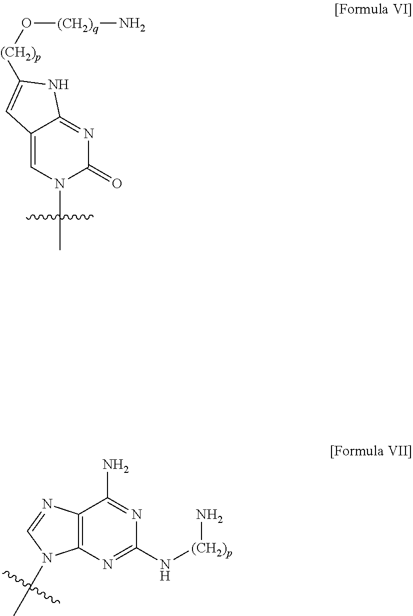

wherein,

p and q are integers, and p is 1 or 6 and q is 2 in ASO 1; and,

“Fethoc-” is the abbreviation for “[2-(9-fluorenyl)ethyl-1-oxy]carbonyl” and “—NH₂” is for non-substituted “-amino” group.

FIG. 3 collectively and unambiguously provides the chemical structures for the PNA monomers abbreviated as A, G, T, C, C(pOq), A(p), A(pOq), G(p), and G(pOq). As discussed in the prior art [PCT/KR2009/001256], C(pOq) is regarded as a “modified cytosine” PNA monomer due to its hybridization for “guanine”. A(p) and A(pOq) are taken as “modified adenine” PNA monomers due to their hybridization for “thymine”.

In order to illustrate the abbreviations employed for such PNA derivatives, the chemical structure of ASO 1 is provided in FIGS. 4.

ASO 1 is equivalent to the DNA sequence of “(5′→3′) TAC-TCA-CCA-TAT-AT” for complementary binding to pre-mRNA. The 14-mer PNA has a 14-mer complementary overlap with the 14-mer sequence marked “bold” and “underlined” within the 30-mer RNA sequence of [(5′→3′) UCCAAGCCAUAUAUG|gugaguauggggaaa] spanning the junction of “exon 5” and “intron 5” in the human MMP-1 pre-mRNA.

In some embodiments, the present invention provides a method of treating diseases or conditions associated with human MMP-1 gene transcription in a subject, comprising administering to the subject the peptide nucleic acid derivative of the present invention or a pharmaceutically acceptable salt thereof.

In some embodiments, the present invention provides a method of treating skin aging in a subject, comprising administering to the subject the peptide nucleic acid derivative of the present invention or a pharmaceutically acceptable salt thereof.

In some embodiments, the present invention provides a pharmaceutical composition for treating diseases or conditions associated with human MMP-1 gene transcription, comprising the peptide nucleic acid derivative of the present invention or a pharmaceutically acceptable salt thereof.

In some embodiments, the present invention provides a cosmetic composition for treating diseases or conditions associated with human MMP-1 gene transcription, comprising the peptide nucleic acid derivative of the present invention or a pharmaceutically acceptable salt thereof.

In some embodiments, the present invention provides a pharmaceutical composition for treating skin aging, comprising the peptide nucleic acid derivative of the present invention or a pharmaceutically acceptable salt thereof.

In some embodiments, the present invention provides a cosmetic composition for treating skin aging, comprising the peptide nucleic acid derivative of the present invention or a pharmaceutically acceptable salt thereof.

Diseases or conditions associated with human MMP-1 gene transcription can be treated by administering a PNA derivative of Formula I or a pharmaceutically acceptable salt thereof.

Diseases or conditions associated with skin aging can be treated by administering a PNA derivative of Formula I or a pharmaceutically acceptable salt thereof.

### BRIEF EXPLANATION OF DRAWINGS

FIGS. 1a-1c. Examples of natural or unnatural (modified) nucleobases selectable for the peptide nucleic acid derivative of Formula I.

FIGS. 2a-2e. Examples of substituents selectable for the peptide nucleic acid derivative of Formula I.

FIG. 3. Chemical structures of PNA monomers with natural or modified nucleobase.

FIG. 4. Chemical structure of “ASO 1”.

FIG. 5. Chemical structures of Fmoc-PNA monomers used to synthesize the PNA derivatives of this invention.

FIGS. 6a-6b. C₁₈-reverse phase HPLC chromatograms of “ASO 1” before and after HPLC purification, respectively.

FIG. 7. ESI-TOF mass spectrum of “ASO 1” purified by C₁₈-RP prep HPLC.

FIG. 8. Inhibition of MMP-1 mRNA Formation by “ASO 1” in HDF (Real-Time qPCR).

FIG. 9a-9b. Inhibition of MMP-1 Protein Expression by “ASO 1” in HDF (Western Blot and Graph for Protein Expression Level Changes).

FIG. 10a-10b. Enhancement of Collagen Protein Expression by “ASO 1” in HDF (Western Blot and Graph for Protein Expression Level Changes).

FIG. 11a-11b. Inhibition of MMP-1 Protein Expression by “ASO 1” in extracellular fluid (Western Blot and Graph for Protein Expression Level Changes).

FIG. 12a-12b. Enhancement of Collagen Protein Expression by “ASO 1” in extracellular fluid (Western Blot and Graph for Protein Expression Level Changes).

FIG. 13. Inhibition of MMP-1 Protein Expression by “ASO 1” in extracellular fluid (ELISA).

FIG. 14. Mammalian pre-mRNA exons and introns.

FIG. 15. Pre-mRNA is processed into mRNA following deletion of introns by a series of complex reactions collectively called “splicing”.

FIG. 16. 3′ splice site and 5′ splice site.

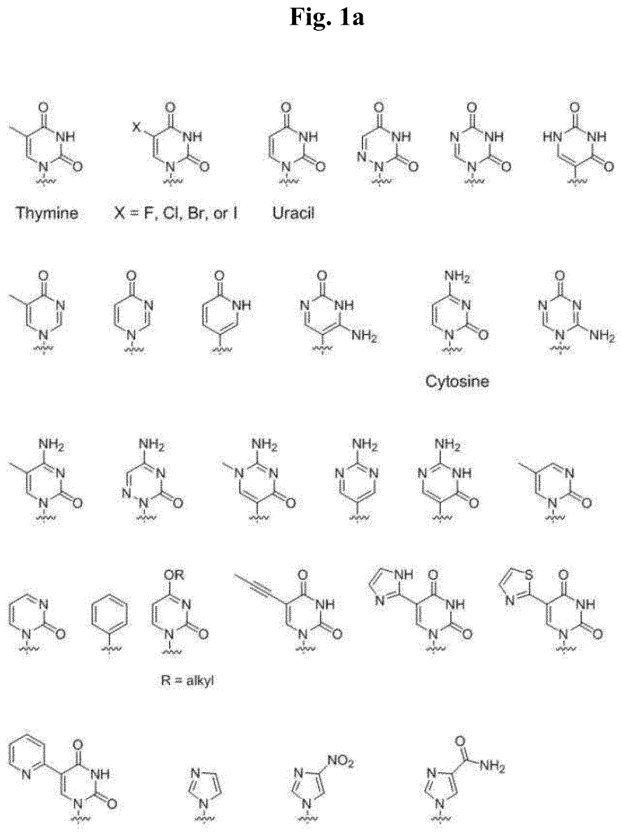

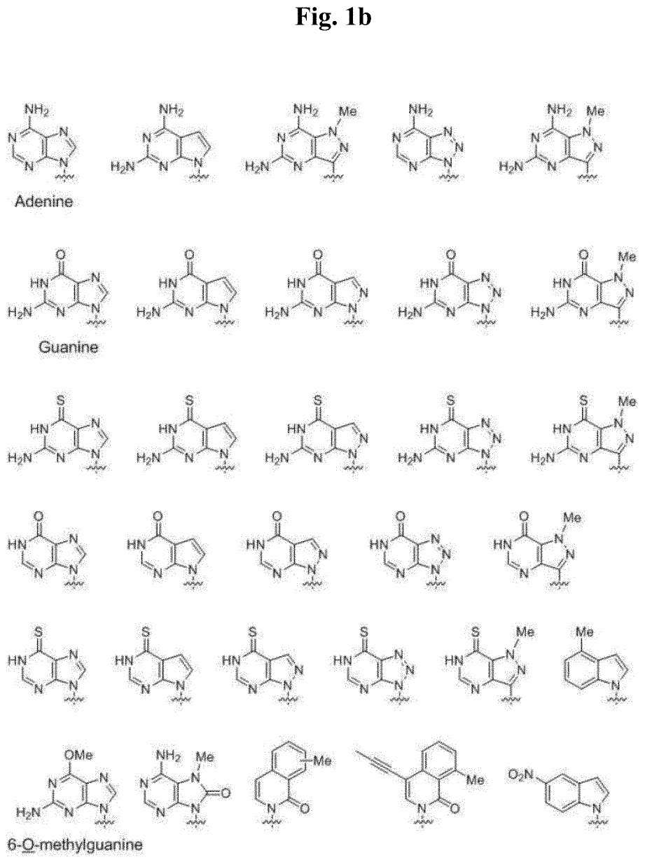

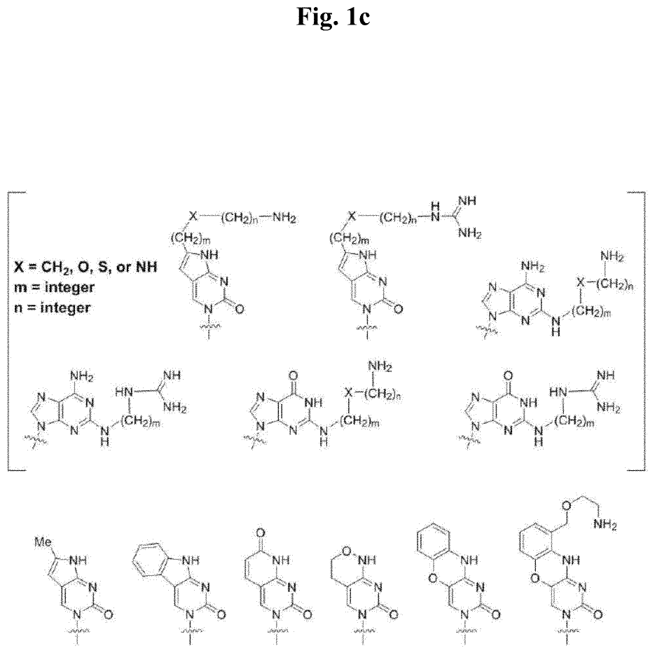

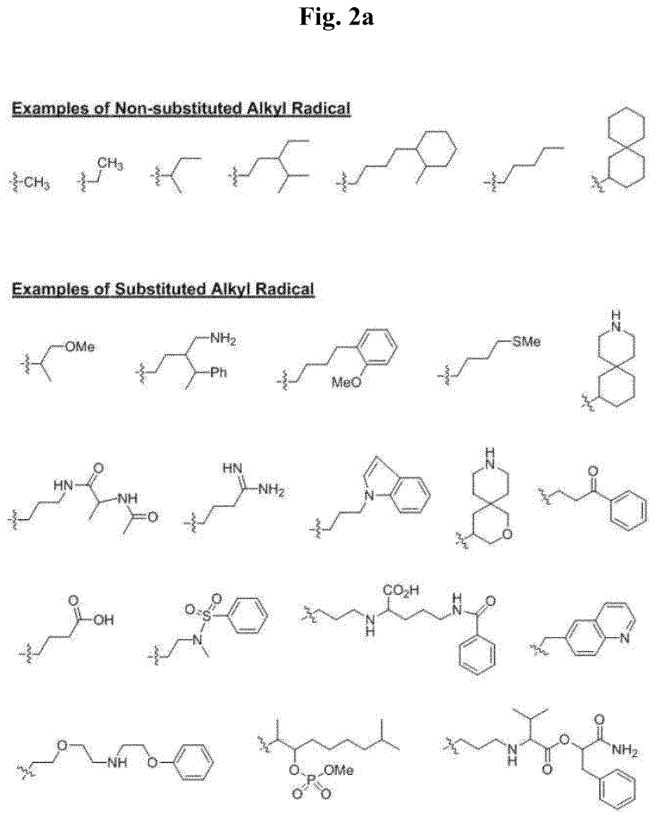

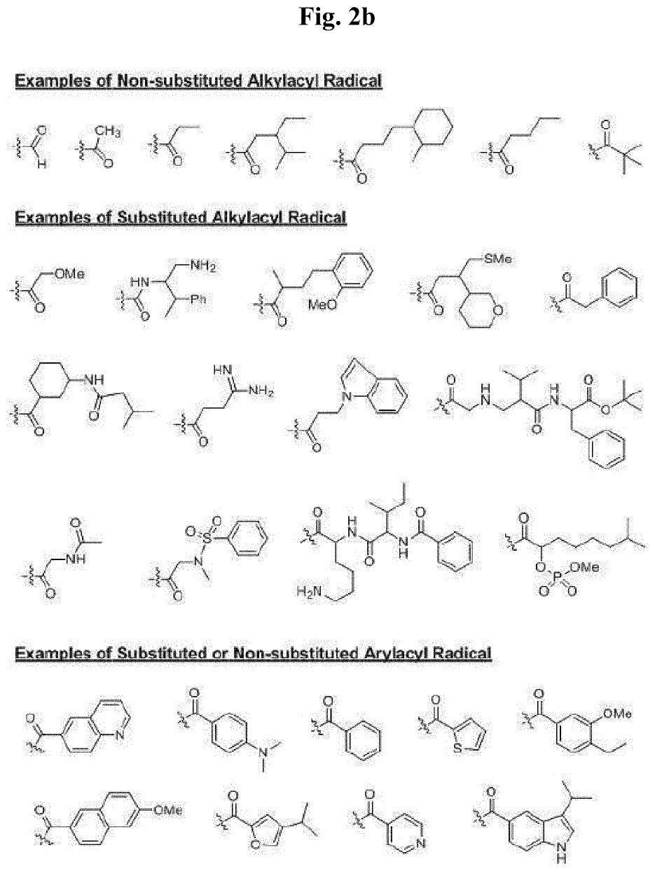

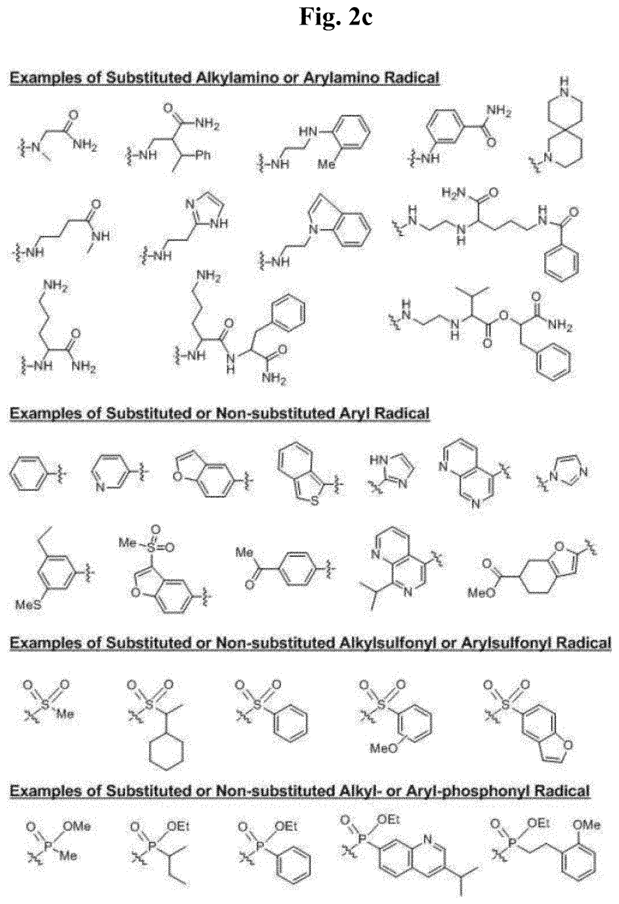

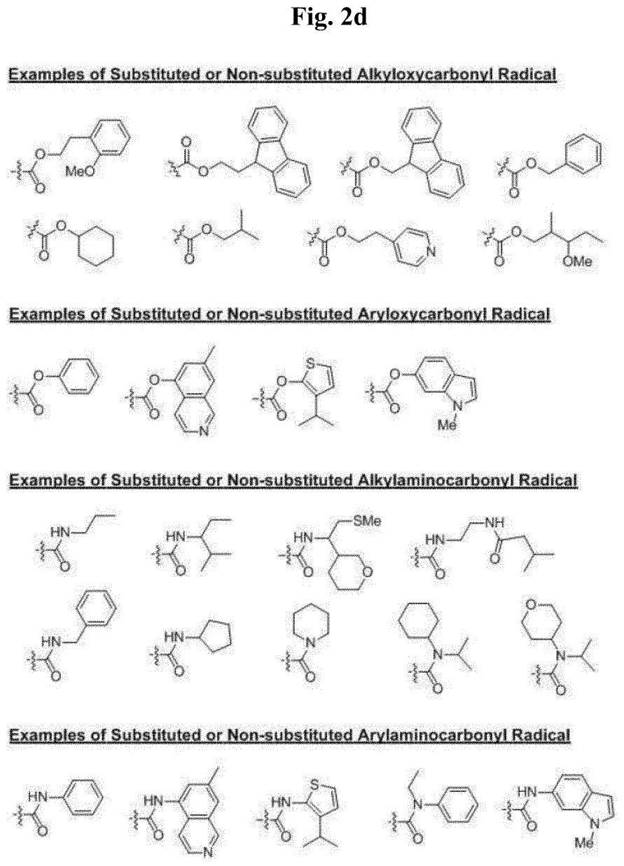

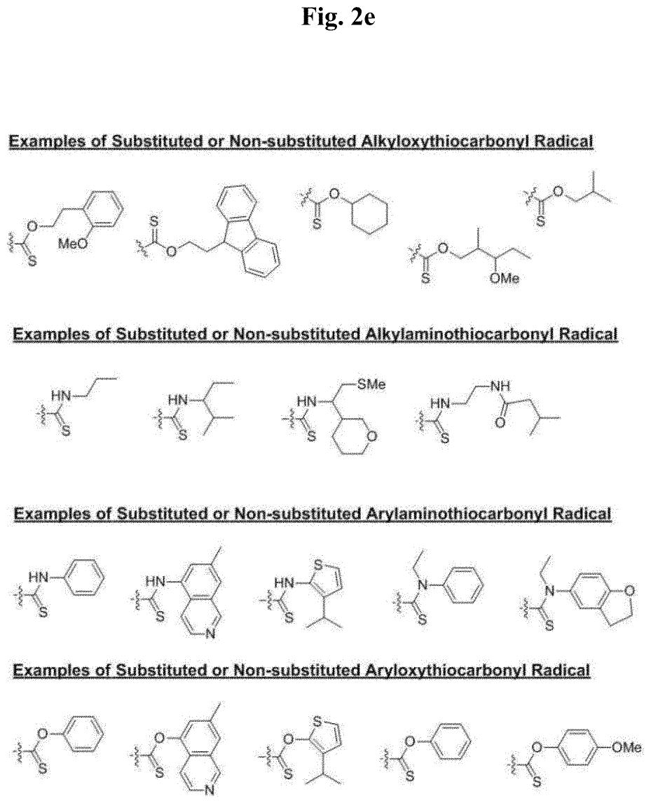

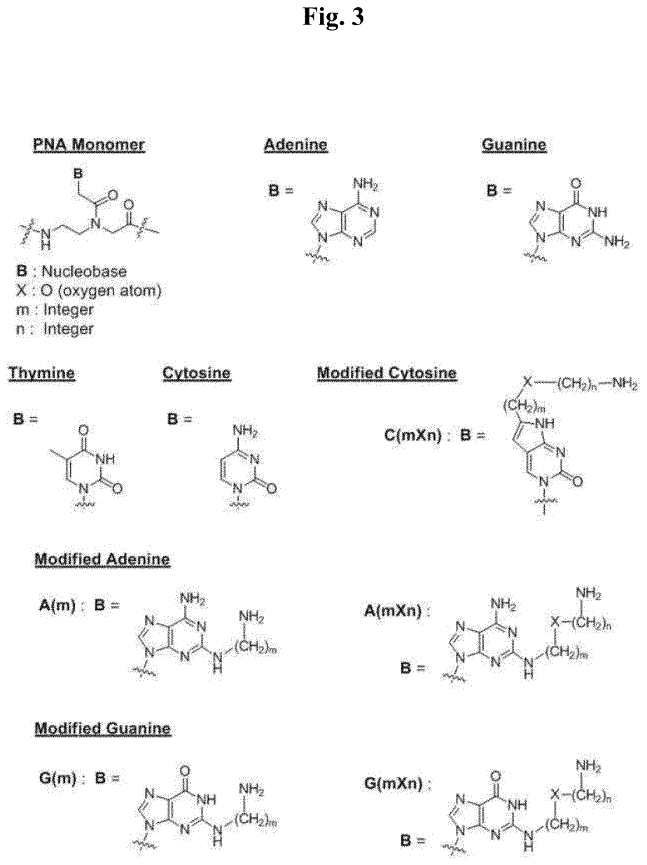

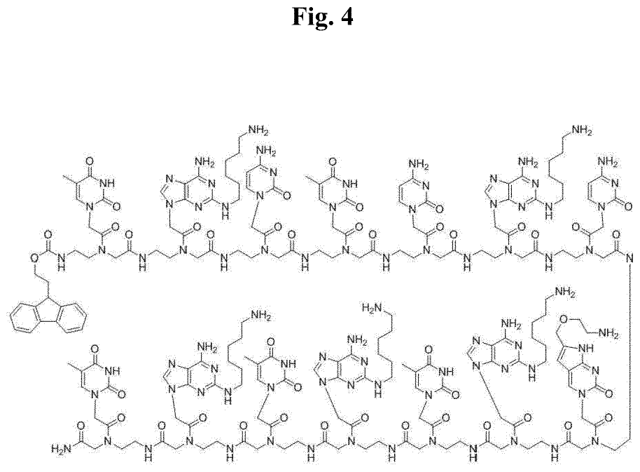

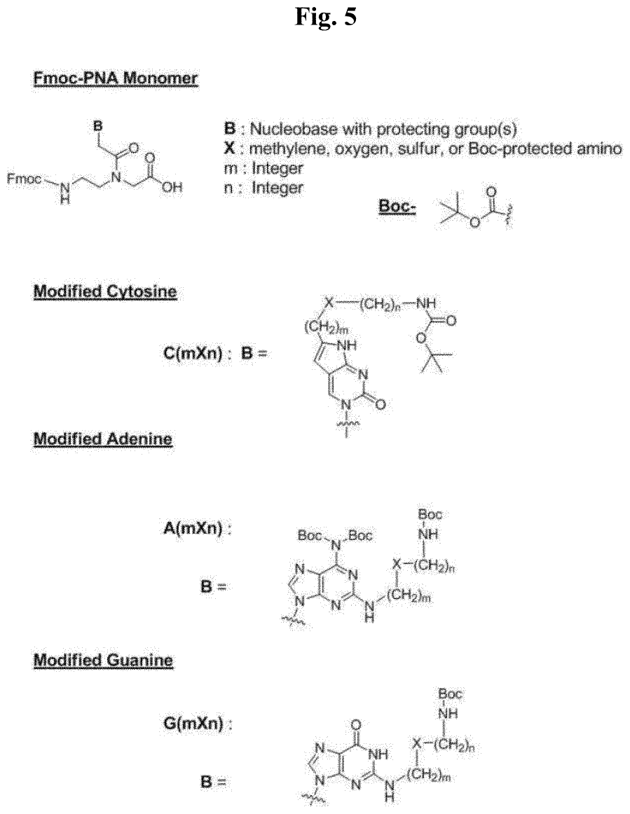

### BEST MODE FOR CARRYING OUT THE INVENTION

General Procedures for Preparation of PNA Oligomers

PNA oligomers were synthesized by solid phase peptide synthesis (SPPS) based on Fmoc-chemistry according to the method disclosed in the prior art [U.S. Pat. No. 6,133,444; WO96/40685] with minor but due modifications. The solid support employed in this study was H-Rink Amide-ChemMatrix purchased from PCAS BioMatrix Inc. (Quebec, Canada). Fmoc-PNA monomers with a modified nucleobase were synthesized as described in the prior art [PCT/KR 2009/001256] or with minor modifications. Such Fmoc-PNA monomers with a modified nucleobase and Fmoc-PNA monomers with a naturally occurring nucleobase were used to synthesize the PNA derivatives of the present invention. PNA oligomers were purified by C₁₈-reverse phase HPLC (water/acetonitrile or water/methanol with 0.1% TFA) and characterized by mass spectrometry including ESI/TOF/MS.

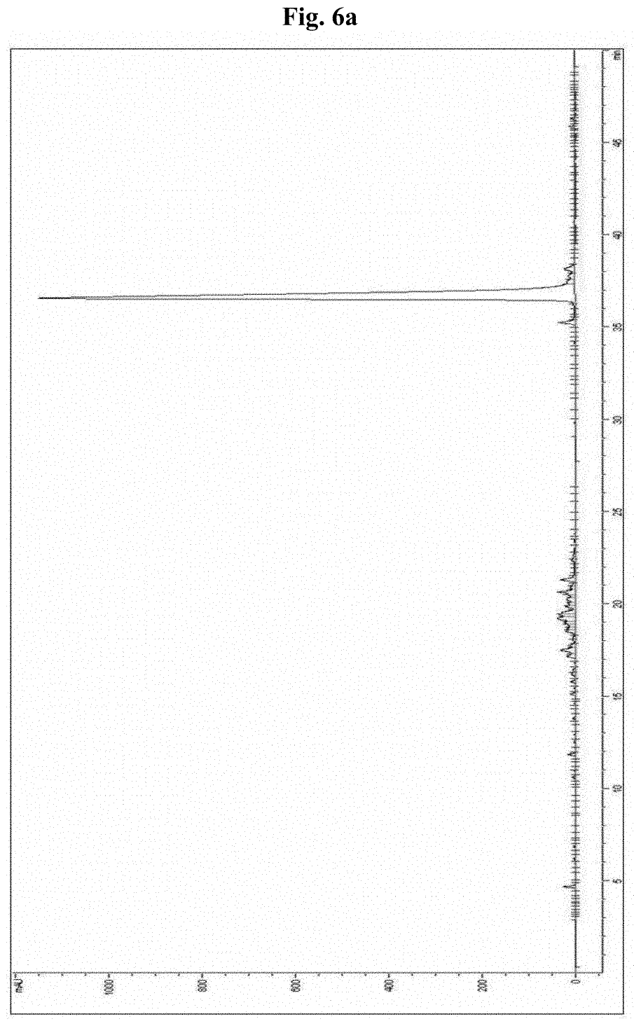

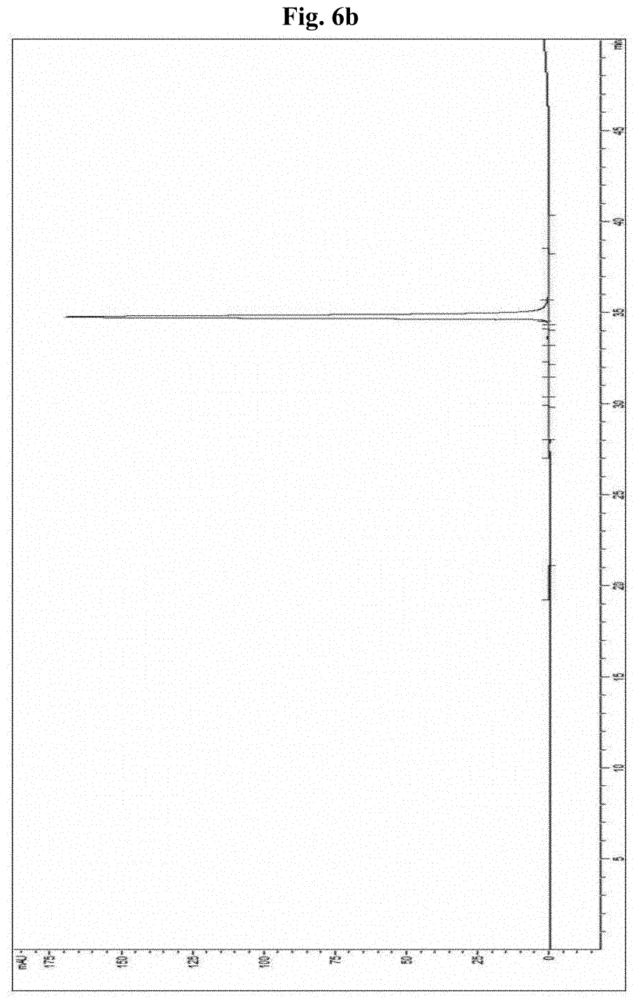

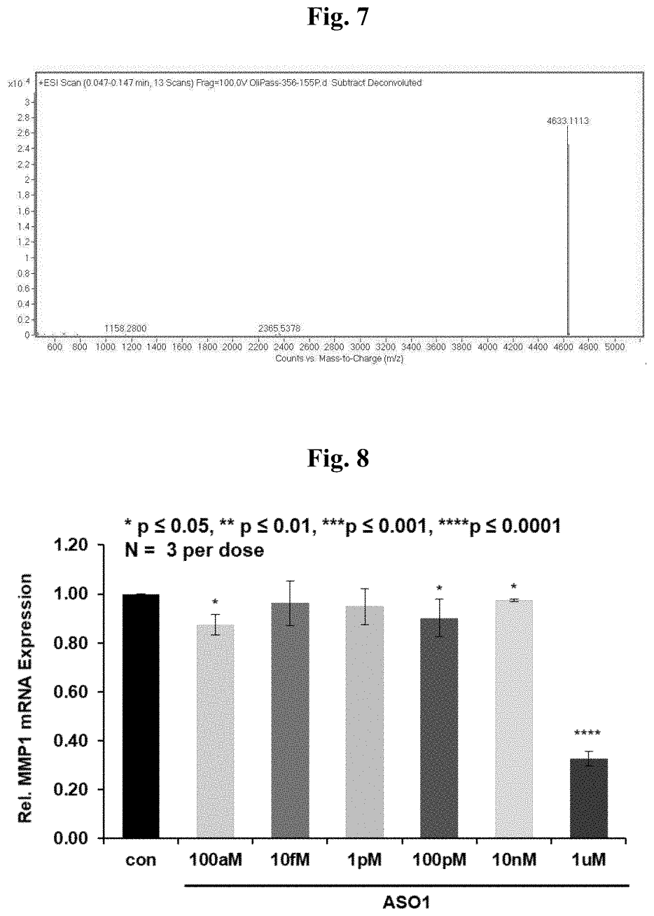

Scheme 1 illustrates a typical monomer elongation cycle adopted in the SPPS of this study, and the synthetic details are provided as below. To a skilled person in the field, however, there are lots of minor variations obviously possible in effectively running such SPPS reactions on an automatic peptide synthesizer or manual peptide synthesizer. Each reaction step in Scheme 1 is briefly provided as follows.

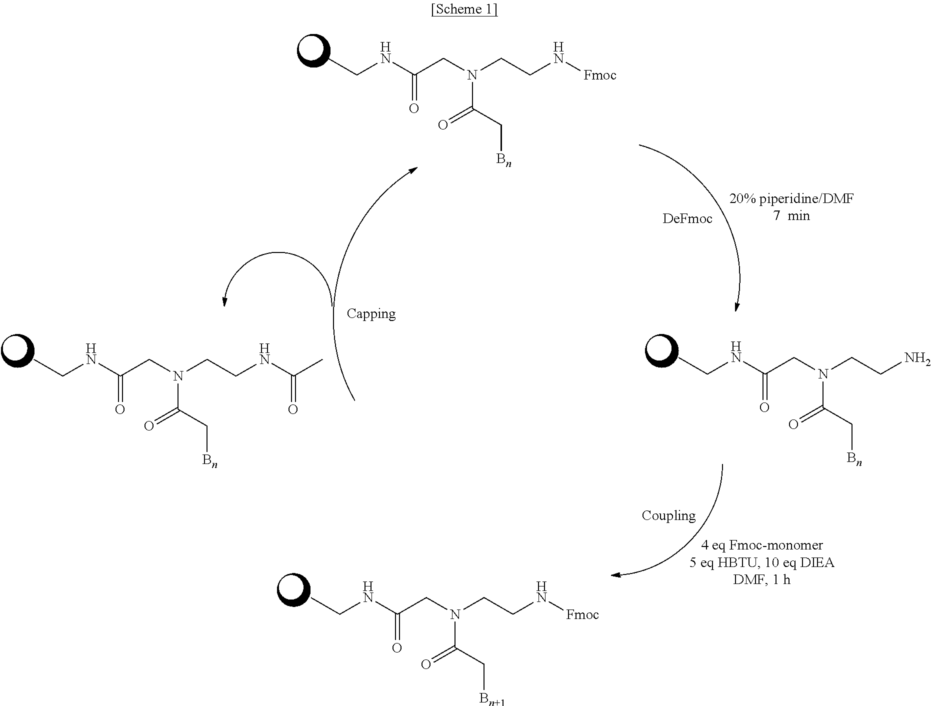

[DeFmoc] The resin was vortexed in 1.5 mL 20% piperidine/DMF for 7 min, and the DeFmoc solution was filtered off. The resin was washed for 30 sec each in series with 1.5 mL MC, 1.5 mL DMF, 1.5 mL MC, 1.5 mL DMF, and 1.5 mL MC. The resulting free amines on the solid support were immediately subjected to coupling with an Fmoc-PNA monomer.

[Coupling with Fmoc-PNA Monomer] The free amines on the solid support were coupled with an Fmoc-PNA monomer as follows. 0.04 mmol of PNA monomer, 0.05 mmol HBTU, and 10 mmol DIEA were incubated for 2 min in 1 mL anhydrous DMF, and added to the resin with free amines. The resin solution was vortexed for 1 hour and the reaction medium was filtered off. Then the resin was washed for 30 sec each in series with 1.5 mL MC, 1.5 mL DMF, and 1.5 mL MC. The chemical structures of Fmoc-PNA monomers with a modified nucleobase used in this invention are provided in FIG. 6. The Fmoc-PNA monomers with a modified nucleobase are provided in FIG. 6 should be taken as examples, and therefore should not be taken to limit the scope of the present invention. A skilled person in the field may easily figure out a number of variations in Fmoc-PNA monomers to synthesize the PNA derivative of Formula I.

[Capping] Following the coupling reaction, the unreacted free amines were capped by shaking for 5 min in 1.5 mL capping solution (5% acetic anhydride and 6% 2,6-leutidine in DMF). Then the capping solution was filtered off and washed for 30 sec each in series with 1.5 mL MC, 1.5 mL DMF, and 1.5 mL MC.

[Introduction of “Fethoc-” Radical in N-Terminus] “Fethoc-” radical was introduced to the N-terminus by reacting the free amine on the resin with “Fethoc-OSu” under basic coupling conditions. The chemical structure of “Fethoc-OSu” [CAS No. 179337-69-0, C₂₀H₁₇NO₅, MW 351.36] is provided as follows.

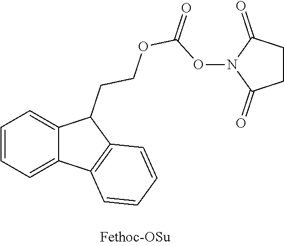 [SMILES: O=C(OCCC1c2ccccc2-c2ccccc21)ON1C(=O)CCC1=O]

[Cleavage from Resin] PNA oligomers bound to the resin were cleaved from the resin by shaking for 3 hours in 1.5 mL cleavage solution (2.5% tri-isopropylsilane and 2.5% water in trifluoroacetic acid). The resin was filtered off and the filtrate was concentrated under reduced pressure. The resulting residue was triturated with diethyl ether and the resulting precipitate was collected by filtration for purification by reverse phase HPLC.

[HPLC Analysis and Purification] Following a cleavage from resin, the crude product of a PNA derivative was purified by C₁₈-reverse phase HPLC eluting water/acetonitrile or water/methanol (gradient method) containing 0.1% TFA. FIGS. 6a and 6b are exemplary HPLC chromatograms for “ASO 1” before and after HPLC purification, respectively.

### Synthetic Examples for PNA Derivative of Formula I

In order to complementarily target the 5′ splice site of “exon 5” in the human MMP-1 pre-mRNA, PNA derivatives of this invention were prepared according to the synthetic procedures provided above or with minor modifications. Provision of such PNA derivatives targeting the human MMP-1 pre-mRNA is to exemplify the PNA derivatives of Formula I, and should not be interpreted to limit the scope of the present invention.

TABLE 1
PNA derivative complementarily targeting the 5′
splice site of “exon 5” in the human MMP-1
pre-mRNA along with structural characterization
data by mass spectrometry.

| PNA | | Exact Mass, m/z | |
|---|---|---|---|
| Example | PNA Sequence (N → C) | theor.^{a} | obs.^{b} |
| ASO 1 | Fethoc-TA(6)C-TCA(6)- | 4631.22 | 4631.22 |
| | CC(1O2)A(6)-TA(6)T- | | |
| | A(6)T-NH₂ | | |

^{a}theoretical exact mass,
^{b}observed exact mass

Table 1 provides PNA derivative complementarily targeting the 5′ splice site of “exon 5” in the human MMP-1 pre-mRNA read out from the human MMP-1 gene [NCBI Reference Sequence: NG_011740] along with structural characterization data by mass spectrometry. Provision of the peptide nucleic acid derivative of the present invention in Table 1 is to exemplify the PNA derivative of Formula I, and should not be interpreted to limit the scope of the present invention.

“ASO 1” has a 14-mer complementary overlap with the 14-mer sequence marked “bold” and “underlined” within the 30-mer RNA sequence of [(5′→3′) UCCAAGCCAUAUAUG|gugaguauggggaaa] spanning the junction of “exon 5” and “intron 5” in the human MMP-1 pre-mRNA. Thus “ASO 1” possesses a 7-mer overlap with “exon 5” and a 7-mer overlap with “intron 5” within the human MMP-1 pre-mRNA.

Binding Affinity of “ASO 1” for Complementary DNA

Tₘ values were determined on a UV/Vis spectrometer as follows. A mixed solution of 4 μM PNA oligomer and 4 μM complementary 14-mer DNA in 4 mL aqueous buffer (pH 7.16, 10 mM sodium phosphate, 100 mM NaCl) in 15 mL polypropylene falcon tube was incubated at 90° C. for a minute and slowly cooled down to ambient temperature. Then the solution was transferred into a 3 mL quartz UV cuvette equipped with an air-tight cap, and subjected to a Tₘ measurement at 260 nm on a UV/Visible spectrophotometer (Agilent 8453). The absorbance changes at 260 nm were recorded with increasing the temperature of cuvette by either 0.5 or 1.0° C. per minute. From the absorbance vs temperature curve, the temperature showing the largest increase rate in absorbance was read out as the melting temperature Tₘ between PNA and DNA. The 14-mer complementary DNAs for Tₘ measurement were purchased from Bioneer (www.bioneer.com, Dajeon, Republic of Korea) and used without further purification.

“ASO 1” showed a Tₘ value of 72.67° C. for the duplex with the 14-mer complementary DNA

### Examples for Biological Activities of PNA Derivatives of Formula I

PNA derivatives in this invention were evaluated for in vitro MMP-1 antisense activities in human dermal fibroblasts (HDF) by use of real-time quantitative polymerase chain reaction (RT qPCR) and so on. The biological examples were provided as examples to illustrate the biological profiles of the PNA derivatives of Formula I, and therefore should not be interpreted to limit the scope of the current invention.

### Example 1. Inhibition of MMP-1 mRNA Formation by “ASO 1” in HDF

“ASO 1” was evaluated by Western blotting for its ability to down-regulate the MMP-1 mRNA formation in HDF as described below.

[Cell Culture & ASO Treatment] HDF cells were maintained in Fibroblast Basal Medium (ATCC PCS-201-030) supplemented with fibroblast growth kit-low serum (ATCC PCS-201-041) and 1% streptomycin/penicillin, which was grown at 37° C. and under 5% CO2 condition. HDF cells (3×10⁵) stabilized for 24 hours in 60 mm culture dish were incubated for 24 hours with “ASO 1” at 0 (negative control) and 100 aM to 1 μM.

[RNA Extraction & cDNA Synthesis] Total RNA was extracted using RNeasy Mini kit (Qiagen, Cat. No. 714106) according to the manufacturer's instructions from ASO 1 treated cells and cDNA was prepared from 400 ng of RNA by use of PrimeScript™ 1^{st} strand cDNA Synthesis Kit (Takara, Cat. No. 6110A). To a mixture of 400 ng of RNA, 1 μl of random hexamer, and 1 μl of dNTP (10 mM) in PCR tube was added DEPC-treated water to a total volume of 10 μl, which was reacted at 65° C. for 5 minutes. cDNA was synthesized by adding 10 μl of PrimeScript RTase to the reaction mixture and reacting at 30° C. for 10 minutes and at 42° C. for 60 minutes, successively.

[Quantitative Real-Time PCR] In order to evaluate the expression level of human MMP-1 mRNA real-time qPCR was performed with synthesized cDNA by use of Taqman probe. The mixture of cDNA, Taqman probe, IQ supermix (BioRad, Cat. No. 170-8862), and nuclease free water in PCR tube was under reaction by use of CFX96 Touch Real-Time system (BioRad) according to the cycle conditions specified as follows: 95° C. for 3 min (primary denaturation) followed by 50 cycles of 10 sec at 95° C. (denaturation), 30 sec at 60° C. (annealing), and 30 sec at 72° C. (polymerization). Fluorescence intensity was measured at the end of every cycle and the result of PCR was evaluated by the melting curve. After the threshold cycle (Ct) of each gene was standardized by that of GAPDH, the change of Ct was compared and analyzed.

[MMP-1 mRNA Decrease by ASO] As can be seen in FIG. 8, compared to control experiment the amount of MMP-1 mRNA reduced were 65% at 1 μM treatment of “ASO 1” and 10 to 15% at 100 aM to 10 nM μM treatment of “ASO 1”, respectively.

### Example 2. Inhibition of MMP-1 Protein Expression by “ASO 1” in HDF

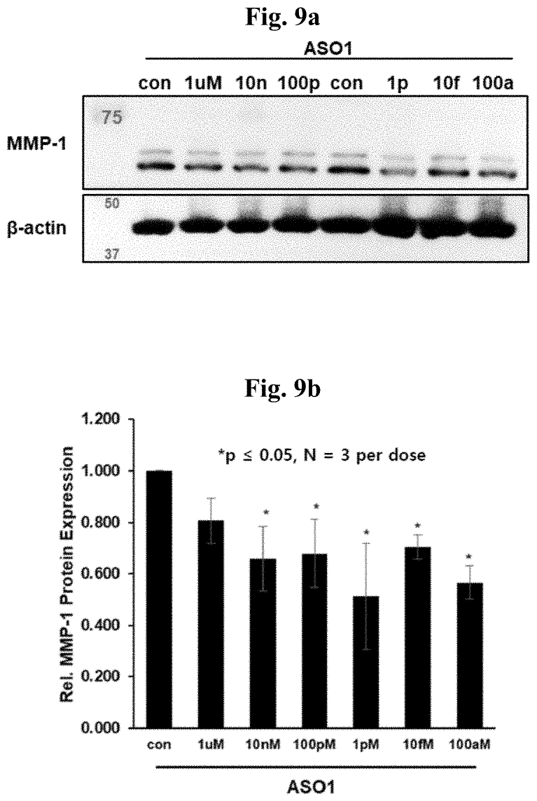

“ASO 1” was evaluated by Western blotting for its ability to down-regulate the MMP-1 protein expression in HDF as described below.

[Western Blotting] HDF cells were grown as Example 1 and 48 hours later cells were washed 2 times with cold PBS (phosphate buffered saline) and dissolved in 50 mM Tris-Cl (pH 7.5), 150 mM NaCl, 1% NP-40, 0.5% sodium deoxycholate, and 0.1% SDS, protease inhibitor. The protein was quantified with BCA solution (Thermo, Cat. No. 23225) and purified by 8% SDS-PAGE Gel. The protein was transferred on PVDF membrane (polyvinylidene fluoride membrane) (Millipore, Cat. No. IPVH00010), which was blocked in skim milk buffer for 1 hour. The membrane was probed with an anti-MMP-1 (SantaCruz, Cat. No. 58377) and anti-β-actin (Sigma, Cat. No. A3854) as a primary antibody, and goat anti-mouse (CST, Cat. No. 7076V) was used as a secondary antibody. SuperSignal™ West Pico (PierAce, USA) was utilized for the detection of chemiluminescent signal and the signal intensity was measured by using Gel Doc system (ATTO). Based on Western blotting results of each bands, the relative expression levels of MMP-1 were quantified with Image-J program.

[Inhibition of MMP-1 Protein Expression by ASO] As shown in FIGS. 9a and 9b, compared to control experiment the amount of MMP-1 protein expression level reduced was 20 to 50% at 100 aM to 1 μM treatment of “ASO 1” Therefore, “ASO 1” was proved to show its ability to down-regulate the MMP-1 protein expression in HDF.

### Example 3. Enhancement of Collagen Protein Expression by “ASO 1” in HDF

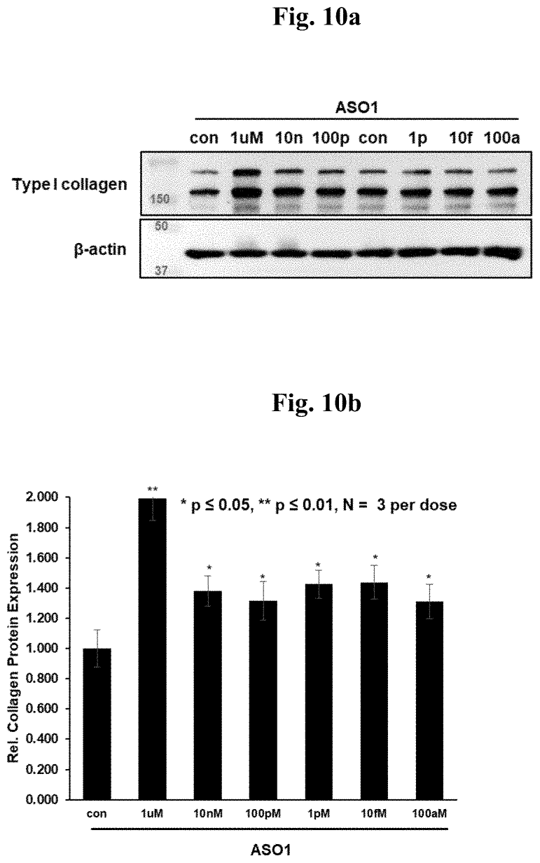

“ASO 1” was evaluated by Western blotting for its ability to up-regulate the type I collagen protein expression in HDF associated with MMP-1 protein expression reduction as described below.

[Western Blotting] HDF cells were grown as Example 1 and 48 hours later cells were washed 2 times with cold PBS (phosphate buffered saline) and dissolved in 50 mM Tris-Cl (pH 7.5), 150 mM NaCl, 1% NP-40, 0.5% sodium deoxycholate, and 0.1% SDS, protease inhibitor. The protein was quantified with BCA solution (Thermo, Cat. No. 23225) and purified by 8% SDS-PAGE Gel. The protein was transferred on PVDF membrane (polyvinylidene fluoride membrane) (Millipore, Cat. No. IPVH00010), which was blocked in skim milk buffer for 1 hour. The membrane was probed with an anti-MMP-1 (SantaCruz, Cat. No. 58377) and anti-β-actin (Sigma, Cat. No. A3854) as a primary antibody, and rabbit anti-goat (Santacruz, Cat. No. 2768) was used as a secondary antibody. SuperSignal™ West Pico (PierAce, USA) was utilized for the detection of chemiluminescent signal and the signal intensity was measured by using Gel Doc system (ATTO). Based on Western blotting results of each bands, the relative expression levels of MMP-1 were quantified with Image-J program.

[Enhancement of Type I Collagen Protein Expression by ASO] As shown in FIGS. 10a and 10b, compared to control experiment the amount of type I collagen protein expression level enhanced was more than 30% at 100 aM to 1 μM treatment of “ASO 1” Therefore, WIMP-1 protein expression reduction induced by “ASO 1” was proved to show its ability to up-regulate the type I collagen protein expression in HDF.

### Example 4. Inhibition of MMP-1 Protein Expression by “ASO 1” in Extracellular Fluid (Western Blotting)

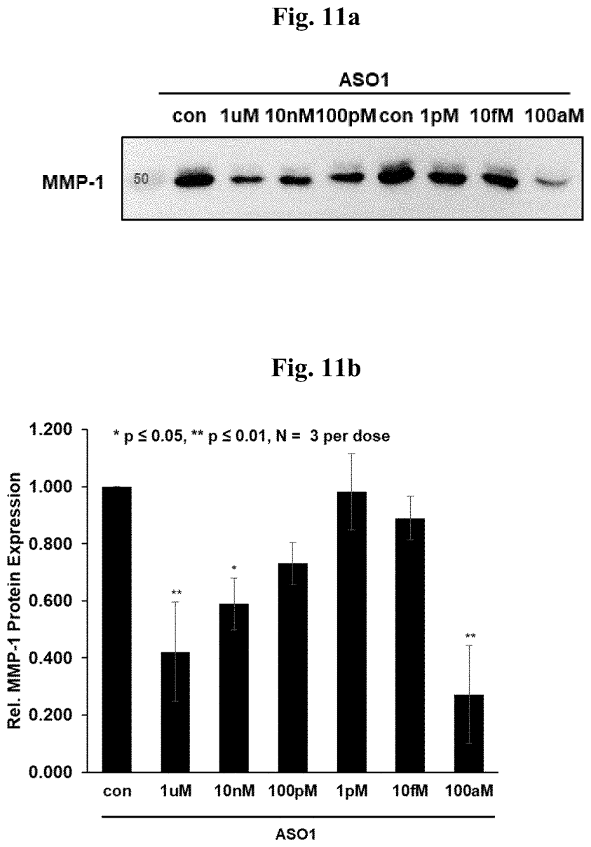

MMP-1 protein expression reduction induced by “ASO 1” in cell, as a result, may affect WIMP-1 protein expression level secreted outside cell. In that sense, “ASO 1” was evaluated by Western blotting for its ability to down-regulate the MMP-1 protein expression in culture fluid of cells at 48 hours after treating “ASO 1” as described below.

[Western Blotting] HDF cells were grown as Example 1 and 48 hours later collected culture fluid of cells was purified by 8% SDS-PAGE Gel. The separated protein was transferred on PVDF membrane (polyvinylidene fluoride membrane) (Millipore, Cat. No. IPVH00010), which was blocked in skim milk buffer for 1 hour. The membrane was probed with an anti-MMP-1 (SantaCruz, Cat. No. 58377) as a primary antibody and goat anti-mouse (CST, Cat. No. 7076V) was used as a secondary antibody. SuperSignal™ West Pico (PierAce, USA) was utilized for the detection of chemiluminescent signal and the signal intensity was measured by using Gel Doc system (ATTO). Based on Western blotting results of each bands, the relative expression levels of MMP-1 were quantified with Image-J program.

[Inhibition of MMP-1 Protein Expression by ASO] As shown in FIGS. 11a and 11b, compared to control experiment the amount of MMP-1 protein expression level reduced was 10 to 60% at 100 μM to 1 μM treatment of “ASO 1” in extracellular fluid. Therefore, “ASO 1” was proved to show its ability to down-regulate the MMP-1 protein expression in extracellular fluid.

### Example 5. Enhancement of Collagen Protein Expression by “ASO 1” in Extracellular Fluid

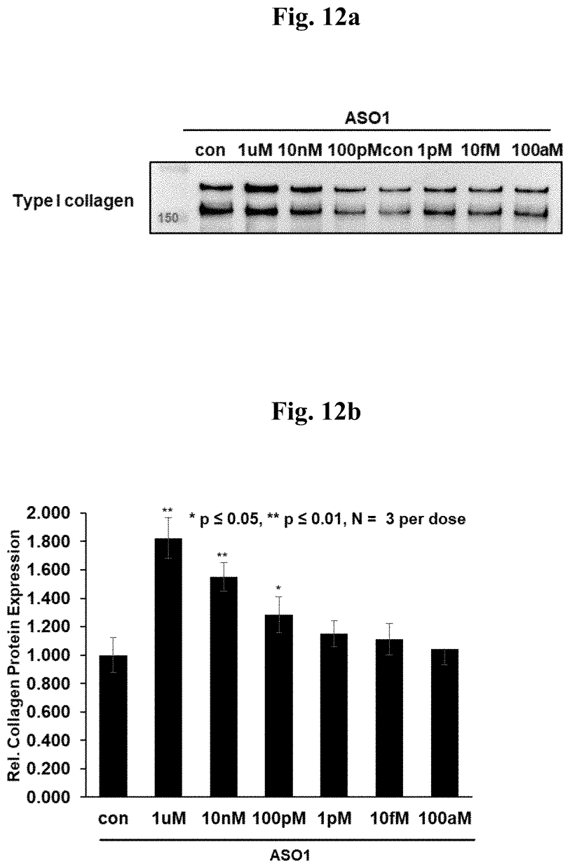

“ASO 1” was evaluated by Western blotting for its ability to up-regulate the type I collagen protein expression in extracellular fluid as described below.

[Western Blotting] HDF cells were grown as Example 1 and 48 hours later collected culture fluid of cells was purified by 8% SDS-PAGE Gel. The separated protein was transferred on PVDF membrane (polyvinylidene fluoride membrane) (Millipore, Cat. No. IPVH00010), which was blocked in skim milk buffer for 1 hour. The membrane was probed with an anti-MMP-1 (SantaCruz, Cat. No. 58377) as a primary antibody and rabbit anti-goat (Santacruz, Cat. No. 2768) was used as a secondary antibody. SuperSignal™ West Pico (PierAce, USA) was utilized for the detection of chemiluminescent signal and the signal Intensity was measured by using Gel Doc system (ATTO). Based on Western blotting results of each bands, the relative expression levels of MMP-1 were quantified with Image-J program.

[Enhancement of Type I Collagen Protein Expression by ASO] As shown in FIGS. 12a and 12b, compared to control experiment the amount of type I collagen protein expression level enhanced was 20% at 1 pM and 80% at 1 μM treatment of “ASO 1”, respectively. Therefore, MMP-1 protein expression reduction induced by “ASO 1” in HDF was proved to show its ability to up-regulate the type I collagen protein expression in extracellular fluid.

### Example 6. Inhibition of MMP-1 Protein Expression by “ASO 1” in Extracellular Fluid (ELISA)

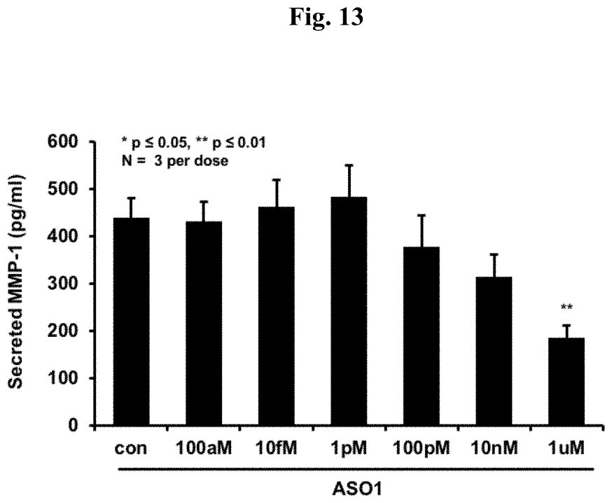

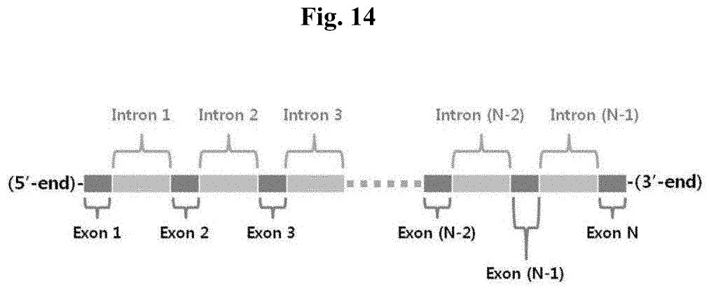

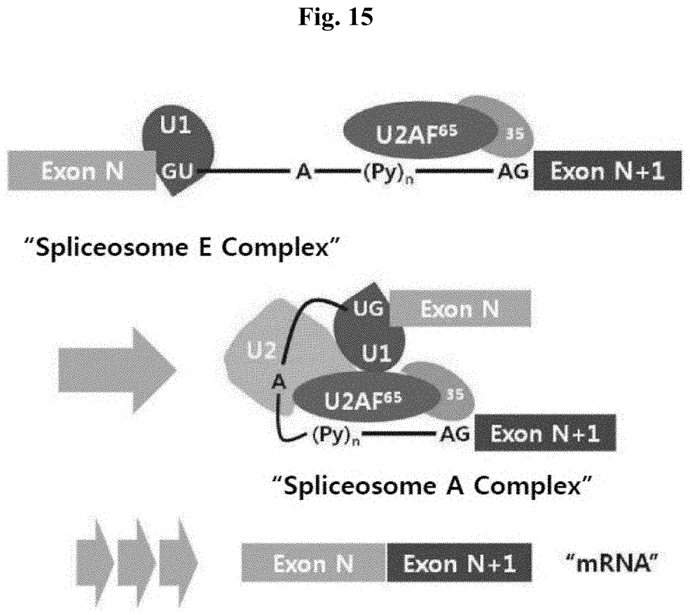

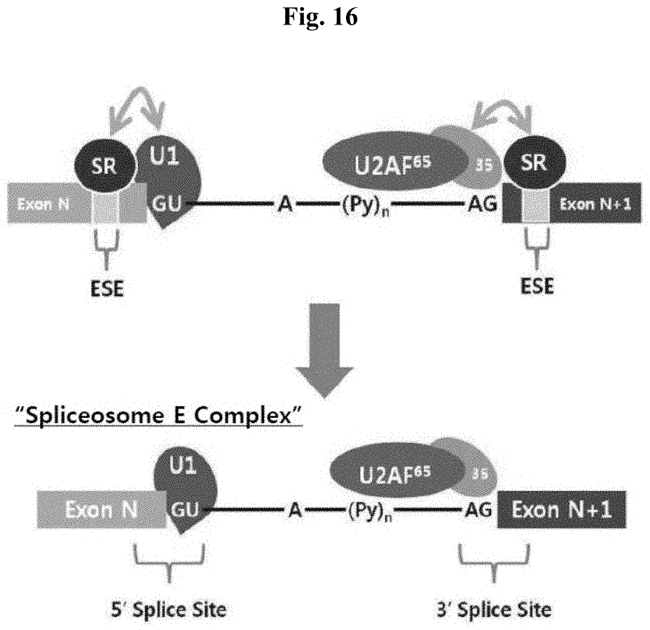

MMP-1 protein expression reduction induced by “ASO 1” in cell, as a result, may affect MMP-1 protein expression level secreted outside cell. In that sense, “ASO 1” was evaluated by enzyme linked immunosorbent assay (ELISA) for its ability to down-regulate the MMP-1 protein expression in culture fluid of cells at 48 hours after treating “ASO 1” as described below.

[ELISA] HDF cells were grown as Example 1 and 48 hours later in collected culture fluid of cells MMP-1 expression level was evaluated through absorbance (Sunrise, TECAN) with human MMP-1 ELISA kit (abcam, Cat. No. ab100603) according to the manufacturer's instruction.

[Inhibition of MMP-1 Protein Expression by ASO] As shown in FIG. 13, compared to control experiment the amount of MMP-1 protein expression level reduced was 15 to 30% at 100 pM to 10 nM and 50% at 1 μM treatment of “ASO 1”, respectively. Therefore, “ASO 1” was proved to show its ability to down-regulate the MMP-1 protein expression in extracellular fluid.

### Example 7. Preparation of Topical Serum Containing Compound of Formula I

(w/w %)

A compound of Formula I, for example “ASO 1” was formulated as a serum for topical application to subjects. The topical serum was prepared as described below. Given that there are lots of variations of topical serum possible, this preparation should be taken as an example and should not be interpreted to limit the scope of the current invention.

| | | | Amount |
|---|---|---|---|
| Part | No. | Substance Name | (w/w %) |
| A | 1 | PEG-40 Hydrogenated Castor Oil | 0.500 |
| | 2 | Ethylhexylglycerin | 0.200 |
| | 3 | Perfume | 0.050 |
| | 4 | Glycerin | 5.000 |
| B | 5 | Butylene Glycol | 7.000 |
| | 6 | Dipropylene Glycol | 2.000 |
| | 7 | 1,2-Hexandiol | 0.200 |
| | 8 | Arginine | 0.150 |
| | 9 | Deionized Water | 58.615 |
| C | 10 | Sodium Hyaluronate | 0.100 |
| | 11 | Acrylates/C10-30 Alkyl Acrylate Crosspolymer | 0.100 |
| | 12 | Carbomer | 0.060 |
| | 13 | Ammonium Acryloyldimethyltaurate/VP | 0.025 |
| | | Copolymer | |
| | 14 | Deionized Water | 23.000 |
| D | 15 | β-glucan | 2.000 |
| | 16 | Biosaccharide Gum-1 | 0.500 |
| | 17 | “ASO 1” 1 pM + Polysorbate 80 0.1% | 0.500 |

| | |
|---|---|
| SUM | 100.000 |

In a separate beaker, the mixed substances of part A and part B at 25° C., respectively, were dissolved. Part A and part B was mixed and emulsified by use of 3,600 rpm homogenizer at 25° C. for 5 minutes. Emulsified part C was filtered through 50 mesh and the filtrate was added to the mixture of part A and B. The resulting mixture was emulsified by use of 3,600 rpm homogenizer at 80° C. for 5 minutes. After addition of part D to the mixture of part A, B, and C, the resulting mixture was emulsified by use of 2,500 rpm homogenizer at 25° C. for 3 minutes. Finally make sure homogeneous dispersion and complete defoamation.

### Example 8. Preparation of Topical Cream Containing Compound of Formula I

(w/w %)

A compound of Formula I, for example “ASO 1” was formulated as a cream for topical application to subjects. The topical cream was prepared as described below. Given that there are lots of variations of topical cream possible, this preparation should be taken as an example and should not be interpreted to limit the scope of the current invention.

In a separate beaker, were dissolved substances of part A at 80° C. and part B at 85° C., respectively. Part A and part B was mixed and emulsified by use of 3,600 rpm homogenizer at 80° C. for 5 minutes. After addition of part C and D to the mixture of part A and B, the resulting mixture was emulsified by use of 3,600 rpm homogenizer at 80° C. for 5 minutes. After addition of part E to the mixture of part A, B, C, and D at 35° C., the resulting mixture was emulsified by use of 3,600 rpm homogenizer at 35° C. for 3 minutes. Finally make sure homogeneous dispersion and complete defoamation at 25° C.

| | | | Amount |
|---|---|---|---|
| Part | No. | Substance Name | (w/w %) |
| A | 1 | Shea Butter | 15.000 |
| | 2 | Simmondsia Chinensis (Jojoba) Seed Oil | 8.000 |
| | 3 | Caprylic/Capric Triglyceride | 6.000 |
| | 4 | Sunflower Seed Oil | 4.000 |
| | 5 | Cetearyl Alcohol | 3.000 |
| | 6 | Glyceryl Stearate | 2.500 |
| | 7 | PEG-100 Stearate | 2.500 |
| | 8 | Macadamia Seed Oil | 2.000 |
| | 9 | Polysorbate 80 | 0.500 |
| B | 10 | 1,3-Propanediol | 2.000 |
| | 11 | Glycerin | 1.000 |
| | 12 | Deionized Water | 51.630 |
| C | 13 | Corn Starch | 0.300 |
| D | 14 | Hydroxyethyl Acrylate/Sodium | 0.300 |
| | | Acryloyldimethyl Tau | |
| E | 15 | 1,2-Hexanediol | 0.300 |
| | 16 | Ethylhexylglycerin | 0.300 |
| | 17 | Tocopheryl Acetate | 0.100 |
| | 18 | Perfume | 0.070 |
| | 19 | “ASO 1” 1 pM + Polysorbate 80 0.1% | 0.500 |

| | |
|---|---|
| SUM | 100.000 |

1. A peptide nucleic acid derivative represented by Formula I, or a pharmaceutically acceptable salt thereof: 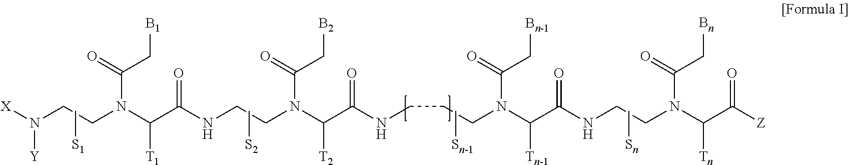
wherein,
n is an integer between 10 and 21;
the compound of Formula I possesses at least a 10-mer complementary overlap with the 16-mer pre-mRNA sequence of [(5′→3′) CAUAUAUGGUGAGUAU] in the human MMP-1 pre-mRNA;
the compound of Formula I is fully complementary to the human MMP-1 pre-mRNA, or partially complementary to the human MMP-1 pre-mRNA with one or two mismatches;
S₁, S₂, . . . , Sₙ₋₁, Sₙ, T₁, T₂, . . . , Tₙ₋₁, and Tₙ independently represent hydrido, deuterido, substituted or non-substituted alkyl, or substituted or non-substituted aryl radical;
X and Y independently represent hydrido, deuterido, formyl [H—C(═O)—], aminocarbonyl [NH₂—C(═O)—], aminothiocarbonyl [NH₂—C(═S)—], substituted or non-substituted alkyl, substituted or non-substituted aryl, substituted or non-substituted alkyloxy, substituted or non-substituted aryloxy, substituted or non-substituted alkylacyl, substituted or non-substituted arylacyl, substituted or non-substituted alkyloxycarbonyl, substituted or non-substituted aryloxycarbonyl, substituted or non-substituted alkylaminocarbonyl, substituted or non-substituted arylaminocarbonyl, substituted or non-substituted alkylaminothiocarbonyl, substituted or non-substituted arylaminothiocarbonyl, substituted or non-substituted alkyloxythiocarbonyl, substituted or non-substituted aryloxythiocarbonyl, substituted or non-substituted alkylsulfonyl, substituted or non-substituted arylsulfonyl, substituted or non-substituted alkylphosphonyl, or substituted or non-substituted arylphosphonyl radical;
Z represents hydrido, deuterido, hydroxy, substituted or non-substituted alkyloxy, substituted or non-substituted aryloxy, substituted or non-substituted amino, substituted or non-substituted alkyl, or substituted or non-substituted aryl radical;
B₁, B₂, . . . , Bₙ₋₁, and Bₙ are independently selected from natural nucleobases including adenine, thymine, guanine, cytosine and uracil, and unnatural nucleobases; and,
at least four of B₁, B₂, . . . , Bₙ₋₁, and Bₙ are independently selected from unnatural nucleobases with a substituted or non-substituted amino radical covalently linked to the nucleobase moiety.

2. The peptide nucleic acid derivative according to claim 1, or a pharmaceutical salt thereof:
wherein,
n is an integer between 10 and 21;
the compound of Formula I possesses at least a 10-mer complementary overlap with the 16-mer pre-mRNA sequence of [(5′→3′) CAUAUAUGGUGAGUAU] in the human MMP-1 pre-mRNA;
the compound of Formula I is fully complementary to the human MMP-1 pre-mRNA, or partially complementary to the human MMP-1 pre-mRNA with one or two mismatches;
S₁, S₂, . . . , Sₙ₋₁, Sₙ, T₁, T₂, . . . , Tₙ₋₁, and Tₙ independently represent hydrido, deuterido radical;
X and Y independently represent hydrido, deuterido, formyl [H—C(═O)—], aminocarbonyl [NH₂—C(═O)—], aminothiocarbonyl [NH₂—C(═S)—], substituted or non-substituted alkyl, substituted or non-substituted aryl, substituted or non-substituted alkyloxy, substituted or non-substituted aryloxy, substituted or non-substituted alkylacyl, substituted or non-substituted arylacyl, substituted or non-substituted alkyloxycarbonyl, substituted or non-substituted aryloxycarbonyl, substituted or non-substituted alkylaminocarbonyl, substituted or non-substituted arylaminocarbonyl, substituted or non-substituted alkylaminothiocarbonyl, substituted or non-substituted arylaminothiocarbonyl, substituted or non-substituted alkyloxythiocarbonyl, substituted or non-substituted aryloxythiocarbonyl, substituted or non-substituted alkylsulfonyl, substituted or non-substituted arylsulfonyl, substituted or non-substituted alkylphosphonyl, or substituted or non-substituted arylphosphonyl radical;
Z represents-hydrido, hydroxy, substituted or non-substituted alkyloxy, substituted or non-substituted aryloxy, or substituted or non-substituted amino radical;
B₁, B₂, . . . , Bₙ₋₁, and Bₙ are independently selected from natural nucleobases including adenine, thymine, guanine, cytosine and uracil, and unnatural nucleobases;
at least four of B₁, B₂, . . . , Bₙ₋₁, and Bₙ are independently selected from unnatural nucleobases represented by Formula II, Formula III, or Formula IV: 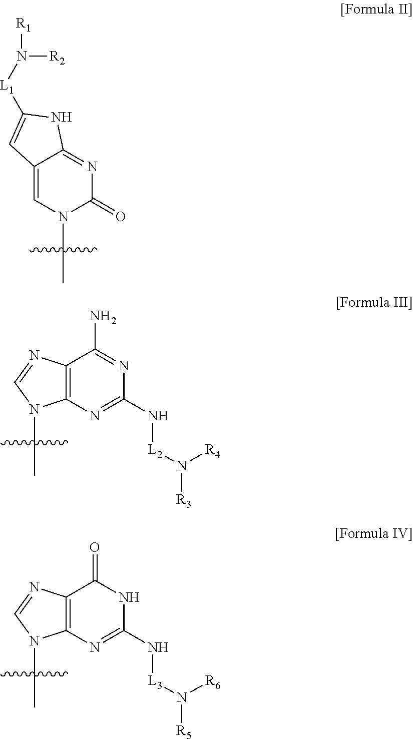
wherein,
R₁, R₂, R₃, R₄, R₅ and R₆ are independently selected from hydrido and substituted or non-substituted alkyl radical;
L₁, L₂ and L₃ are a covalent linker represented by Formula V covalently linking the basic amino group to the nucleobase moiety: 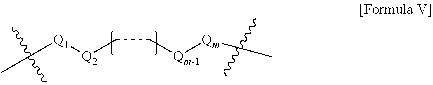
wherein,
Q₁ and Qₘ are substituted or non-substituted methylene (—CH₂—) radical, and Qₘ is directly linked to the basic amino group;
Q₂, Q₃, . . . , and Qₘ₋₁ are independently selected from substituted or non-substituted methylene, oxygen (—O—), sulfur (—S—), and substituted or non-substituted amino radical [—N(H)—, or —N(substituent)-]; and,
m is an integer between 1 and 15.

3. The peptide nucleic acid derivative according to claim 2, or a pharmaceutical salt thereof:
wherein,
n is an integer between 11 and 16;
the compound of Formula I possesses at least a 10-mer complementary overlap with the 16-mer pre-mRNA sequence of [(5′→3′) CAUAUAUGGUGAGUAU] in the human MMP-1 pre-mRNA;
the compound of Formula I is fully complementary to the human MMP-1 pre-mRNA;
S₁, S₂, . . . , Sₙ₋₁, Sₙ, T₁, T₂, . . . , Tₙ₋₁, and Tₙ are hydrido radical;
X and Y independently represent hydrido, substituted or non-substituted alkylacyl, or substituted or non-substituted alkyloxycarbonyl radical;
Z represents substituted or non-substituted amino radical;
B₁, B₂, . . . , Bₙ₋₁, and Bₙ are independently selected from natural nucleobases including adenine, thymine, guanine, cytosine and uracil, and unnatural nucleobases;
at least five of B₁, B₂, . . . , Bₙ₋₁, and Bₙ are independently selected from unnatural nucleobases represented by Formula II, Formula III, or Formula IV;
R₁, R₂, R₃, R₄, R₅ and R₆ are hydrido radical;
Q₁ and Qₘ are methylene radical, and Qₘ is directly linked to the basic amino group;
Q₂, Q₃, . . . , and Qₘ₋₁ are independently selected from methylene and oxygen radical; and,
m is an integer between 1 and 9.

4. The peptide nucleic acid derivative according to claim 3, or a pharmaceutical salt thereof:
wherein,
n is an integer between 11 and 16;
the compound of Formula I possesses at least a 10-mer complementary overlap with the 16-mer pre-mRNA sequence of [(5′→3′) CAUAUAUGGUGAGUAU] in the human MMP-1 pre-mRNA;
the compound of Formula I is fully complementary to the human MMP-1 pre-mRNA;
S₁, S₂, . . . , Sₙ₋₁, Sₙ, T₁, T₂, . . . , Tₙ₋₁, and Tₙ are hydrido radical;
X is hydrido radical;
Y represents substituted or non-substituted alkyloxycarbonyl radical;
Z represents substituted or non-substituted amino radical;
B₁, B₂, . . . , Bₙ₋₁, and Bₙ are independently selected from natural nucleobases including adenine, thymine, guanine, cytosine and uracil, and unnatural nucleobases;
at least five of B₁, B₂, . . . , Bₙ₋₁, and Bₙ are independently selected from unnatural nucleobases represented by Formula II, Formula III, or Formula IV;
R₁, R₂, R₃, R₄, R₅ and R₆ are hydrido radical;
L₁ represents —(CH₂)₂—O—(CH₂)₂—, —CH₂—O—(CH₂)₂, —CH₂—O—(CH₂)₃—, —CH₂—O—(CH₂)₄—, or —CH₂—O—(CH₂)₅— with the right end is directly linked to the basic amino group; and,
L₂ and L₃ are independently selected from —(CH₂)₂—O—(CH₂)₂—, —(CH₂)₃—O—(CH₂)₂—, —(CH₂)₂—O—(CH₂)₃—, —(CH₂)₂—, —(CH₂)₃—, —(CH₂)₄—, —(CH₂)₅—, —(CH₂)₆—, —(CH₂)₇—, and —(CH₂)₈— with the right end is directly linked to the basic amino group.

5. The peptide nucleic acid derivative according to claim 4, which is a peptide nucleic acid derivative provided below, or a pharmaceutically acceptable salt thereof:
(N→C) Fethoc-TA(6)C-TCA(6)-CC(102)A(6)-TA(6)T-A(6)T-NH₂
wherein,
A, T, and C are monomers of peptide nucleic acid with a natural nucleobase of adenine, thymine, and cytosine, respectively;
C(pOq) and A(p) are monomers of peptide nucleic acid with an unnatural nucleobase represented by Formula VI and Formula VII, respectively; 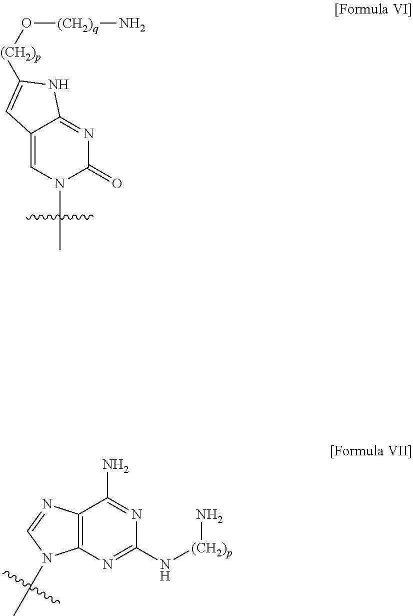
wherein,
p and q are integers and p is 1 or 6, and q is 2; and,
“Fethoc-” is the abbreviation for “[2-(9-fluorenyl)ethyl-1-oxy]carbonyl”.

6. A method to treat diseases or conditions associated with the human MMP-1 gene transcription, comprising the administration of the peptide nucleic acid derivative according to claim 1, or a pharmaceutically acceptable salt thereof to a subject.

7. A method to treat skin aging, comprising the administration of the peptide nucleic acid derivative according to claim 1, or a pharmaceutically acceptable salt thereof to a subject.

8. A pharmaceutical composition for treating diseases or conditions associated with human MMP-1 gene transcription, comprising the peptide nucleic acid derivative according to claim 1, or a pharmaceutically acceptable salt thereof.

9. A cosmetic composition for treating diseases or conditions associated with human MMP-1 gene transcription, comprising the peptide nucleic acid derivative according to claim 1, or a pharmaceutically acceptable salt thereof.

10. A pharmaceutical composition for treating skin aging, comprising the peptide nucleic acid derivative according to claim 1, or a pharmaceutically acceptable salt thereof.

11. A cosmetic composition for treating skin aging, comprising the peptide nucleic acid derivative according to claim 1, or a pharmaceutically acceptable salt thereof.
